(19) United States
(12) Patent Application Publication
(10) Pub. No.: US 2015/0322402 A1
(43) Pub. Date: Nov. 12, 2015

(54) Syntrophic co-culture of anaerobic microorganism for production of n-butanol from syngas

(71) Applicant: Coskata, Inc., Warrenville, IL (US)

(72) Inventors: Rathin Datta, Chicago, IL (US); Andrew Reeves, Chicago, IL (US)

(21) Appl. No.: 14/812,182

(22) Filed: Jul. 29, 2015

Related U.S. Application Data
(63) Continuation of application No. 13/745,177, filed on Jan. 18, 2013, now abandoned.

(51) Int. Cl. C12N 1/20; C12P 7/16

(52) U.S. Cl. CPC C12N 1/20; C12P 7/16

(57) ABSTRACT

This invention provides compositions for the production of butanol. Specifically, the compositions of the present invention use syntrophic co-cultures for the production of butanol from syngas.

This application claims the benefit of U.S. patent application Ser. No. 13/745,177, filed on Jan. 18, 2013 as a continuation, the disclosure of which is explicitly incorporated by reference herein.

### FIELD OF THE INVENTION

The invention provides a composition for the production of n-butanol and other C4-containing products from syngas using a syntrophic co-culture of anaerobic microorganisms.

### BACKGROUND OF THE INVENTION

Butanol is an important industrial chemical with a wide range of applications. It can be used as a motor fuel particularly in combination with gasoline to which it can be added in all proportions. Isobutanol can also be used a precursor to Methyl Tertiary Butyl Ether (MTBE). Currently the world production of n-Butanol is 3.5 million tons/yr. (7.7 billion lb/yr). Furthermore, conversion of alcohols to long chain linear hydrocarbons that would be suitable for jet fuel use are being developed and demonstrated, which could further increase the demand for n-Butanol (The Naval Air Warfare Center-Weapons Division, (2012) Cobalt and Abermarle). Fermentation of carbohydrates to acetone, butanol and ethanol (ABE) is well known and was commercially practiced worldwide from around 1915 to 1955 (Beesch, S.C. (1953) A Microbiological process Report-Applied Microbiology, 1, 85-95). With the advent of petrochemical processes and low cost petrochemical feedstocks the carbohydrate based processes became unattractive and were discontinued.

Further development and modernization of the ABE process was undertaken by several organizations. In the mid-1980s the Corn Products Corporation developed asporogenic strains and a multi-staged fermentation process that considerably improved the process economics (Marlatt, J. A. and R. Datta, (1986) Acetone-Butanol Fermentation Process, Biotechnology Progress (1986) 2, 1.23-28). Currently, two companies, Gevo and Butamax are engaged in conversion of several ethanol plants using recombinant microorganisms to produce iso-butanol for new chemical uses. See U.S. Pat. No. 8,017,375 and U.S. Pat. No. 7,851,188. In all of these developments the primary feedstock is carbohydrate, primarily starch from corn.

The limitations for carbohydrate feedstocks are well known and some are fundamental. Starch and sugars from agricultural crops run into competing issues of food vs. energy/chemical production as well as the cost of the feedstocks and their availability. For lignocellulosic feedstocks such as woody biomass, grasses etc. the cost and yield from pretreatment and hydrolysis processes are very limiting. For example, typical woody biomass contains 50% cellulose while the remainder consists of hemicelluloses, lignin and other fractions. The chemical energy content of the fermentable fractions is often less than 50% of that of the feedstock, putting fundamental limitations on product yield.

Attempts have been made to improve the alcohol yield of bacterium that ferment a variety of sugars to acetate and butyrate. The art has sought to employ recombinant techniques to transform bacterium such as C. acetobutylicum (Green et al. (1996) Genetic manipulation of acid formation pathways, Green et al. Microbiology), 142, 2079-2086) and C. tyrobutyricum (X. Liu et al. (2006) Construction and Characterization of ack Deleted Mutant of Clostridium tyrobutyricum, Biotechnology Pref., 22, 1265-1275). However, such techniques have only resulted in transformation occurring at low frequencies.

Several microorganisms are able to use one-carbon compounds as carbon source and some even as an energy source. Synthesis gas is a common substrate for supplying the one carbon compounds such as CO and CO₂ as well as hydrogen. Synthesis gas can be produced by gasification of the whole biomass source without the need to unlock certain fractions. Synthesis gas can also be produced from other feedstocks via gasification of: (i) coal, (ii) municipal waste (iii) plastic waste, (iv) petcoke and (v) liquid residues from refineries or from the paper industry (black liquor). Synthesis gas can also be produced from natural gas via steam reforming or autothermal reforming (partial oxidation). When the syngas source is biomass, gasification technology converts all the components of the feedstock primarily to a mixture of CO, H₂, CO₂ and some residual CH₄, typically with 75 to 80% cold gas efficiency i.e. 75 to 80% of the chemical energy of the feedstock is available for further chemical or biological conversion to target products. The rest of the energy is available as heat that can be used to generate steam to provide some or all of the process energy required. Furthermore, a wide range of feedstocks, both renewable such as woody biomass, agricultural residues, municipal wastes etc. or non-renewable such as natural gas, can be gasified to produce these primary components.

Natural gas can be economically reformed to syngas with a wide variety of technologies using steam, oxygen, air or combinations thereof. This syngas has very good cold gas efficiency of approximately 85% to produce CO, H₂ and CO₂ with a wide range of target compositions.

Hence, syngas is a very economical feedstock that can be derived from a wide range of raw materials both renewable and non-renewable. Thus conversion of syngas to butanol with high yield and concentrations would lead to economical production of this important chemical.

The ability of anaerobic bacteria to produce n-butanol from the primary syngas components CO and H₂/CO₂ was discovered and reported in 1990/1991 by a team from the Michigan Biotechnology Institute, (A. Grethlein et al. (1991) Evidence of n-Butanol Production from Carbon Monoxide, Journal of Fermentation and Bioengineering, 72, 1, 58-60); (Grethlien et al. (1990) Continuous Production of Mixed Alcohols and Acids from Carbon Monoxide, Journal of Fermentation and Bioengineering, 24-25(1):875-885). Later, other organizations such as University of Oklahoma and Oklahoma State University also isolated new organisms namely Clostridium carboxydivorans that also showed such conversion and n-butanol production (J. S. Liou et al. (2005) Clostridium carboxidivorans sp. nov. a solvent producing clostridium International Journal of Systematic and Evolutionary Microbiology 55(5):2085-2091). Subsequent fermentation development with these and other organisms in single culture fermentations have not been very successful—the n-butanol concentrations were achieved in the range of approximately 3 g/liter and the yield ranged from 20 to 45% of theoretical (% electrons to product) (see previous three references and Guilaume Bruant et al. (2010) Genomic Analysis of Carbon Monoxide Utilization and Butanol Production by Clostridium carboxidivorans, PLoS One, 5(9)). For a commercially successful process, the n-butanol concentration should be in the range of 8-10 g/liter and the yield should be in the 80% range, otherwise processing and separations costs become unattractive.

To overcome these barriers multi-stage fermentations with two or more organisms such as Butyribacterium methylotrophicum and Clostridium acetobutylicum have been proposed (Worden et al. (1991) Production of butanol and ethanol from synthesis gas via fermentation, Fuel, 70, 6154-619). The former would produce butyric acid and butanol at low concentrations from syngas and the latter would uptake these while converting carbohydrates to produce more butanol. Since C. acetobutylicum strains are able to produce 15 g/liter butanol the separations process would be viable. Such a combination could provide some increases in yield and product recovery, but it would be very cumbersome requiring two different types of feedstocks, syngas and carbohydrates as well as separate bioreactors one for gas conversion and another for carbohydrate conversion. Furthermore, in this scheme the carbohydrate feeding the Clostridium acetobutylicum is the primary feedstock and not the more economical syngas fed to the Butyribacterium methylotrophicum and all the limitations of carbohydrate feedstocks described above will be prevalent.

A more efficient conversion of syngas takes place when converting it to ethanol and acetate. The biochemical pathway of such synthesis gas conversion is described by the Wood-Ljungdahl Pathway. Fermentation of syngas to ethanol and acetate offers several advantages such as high specificity of the biocatalysts, lower energy costs (because of low pressure and low temperature bioconversion conditions), greater resistance to biocatalyst poisoning and nearly no constraint for a preset H₂ to CO ratio (M. Bredwell et al. (1999) Reactor design issues for synthesis-gas fermentations, Biotechnology Progress 15, 834-844); (Klasson et al. (1992), Biological conversion of synthesis gas into fuels”, International Journal of Hydrogen Energy 17, p. 281). Acetogens are a group of anaerobic bacteria able to convert syngas components, like CO, CO₂ and H₂ to acetate and ethanol via the reductive acetyl-CoA or the Wood-Ljungdahl pathway.

Several anaerobic bacteria have been isolated that have the ability to ferment syngas to ethanol, acetic acid and other useful end products. Clostridium ljungdahlii and Clostridium autoethanogenum, were two of the first known organisms to convert CO, CO₂ and H₂ to ethanol and acetic acid. Commonly known as homoacetogens, these microorganisms have the ability to reduce CO2 to acetate in order to produce required energy and to produce cell mass. The overall stoichiometry for the synthesis of ethanol using three different combinations of syngas components is as follows (J. Vega et al. (1989) The Biological Production of Ethanol from Synthesis Gas, Applied Biochemistry and Biotechnology, 20-1, p. 781):

6CO+3H₂O→CH₃CH₂OH+4CO₂

2CO₂+6H₂→CH₃CH₂OH+3H₂O

6CO+6H₂→2CH₃CH₂OH+2CO₂

The primary product produced by the fermentation of CO and/or H₂ and CO₂ by homoacetogens is ethanol principally according to the first two of the previously given reactions. Homoacetogens may also produce acetate. Acetate production occurs via the following reactions:

4CO+2H₂O→CH₃COOH+2CO₂

4H₂+2CO₂→CH₃COOH+2H₂O

Clostridium ljungdahlii, one of the first autotrophic microorganisms known to ferment synthesis gas to ethanol was isolated in 1987, as a homoacetogen it favors the production of acetate during its active growth phase (acetogenesis)) while ethanol is produced primarily as a non-growth-related product (solventogenesis) (K. Klasson et al. (1992) Biological conversion of synthesis gas into fuels, International Journal of Hydrogen Energy 17, p. 281).

Clostridium autoethanogenum is a strictly anaerobic, gram-positive, spore-forming, rod-like, motile bacterium which metabolizes CO to form ethanol, acetate and CO₂ as end products, beside it ability to use CO₂ and H₂, pyruvate, xylose, arabinose, fructose, rhamnose and L-glutamate as substrates (J. Abrini, H. Naveau, E. Nyns,), “Clostridium autoethanogenum, Sp-Nov, an Anaerobic Bacterium That Produces Ethanol from Carbon-Monoxide”, Archives of Microbiology, 161(4), p. 345, 1994).

Anaerobic acetogenic microorganisms offer a viable route to convert waste gases, such as syngas, to useful products, such as ethanol, via a fermentation process. Such bacteria catalyze the conversion of H₂ and CO₂ and/or CO to acids and/or alcohols with higher specificity, higher yields and lower energy costs than can be attained by traditional production processes. While many of the anaerobic microorganisms utilized in the fermentation of ethanol also produce butanol as a secondary product, to date, no single anaerobic microorganism has been described that can utilize the syngas fermentation process to produce high yields of butanol.

Therefore a need in the art remains for syntophic co-cultures using microorganisms in the production of butanol using syngas as the primary fermentation substrate.

### SUMMARY OF THE INVENTION

Provided herein is a microorganism co-culture for the conversion of at least one of CO or CO₂ and H₂ to butanol said co-culture comprising two or more microorganisms collectively having a nucleotide sequence identity at least 95% identical to SEQ ID No. 1 and a nucleotide sequence identity at least 70% identical to SEQ ID No. 2 or at least 65% identical to SEQ. ID No. 3. The new syntrophic co-culture of anaerobic microorganisms is defined by a unique set of nucleotide sequences and can produce butanol from a non-food substrate of CO or CO₂ and H₂ at much higher concentrations than previous methods for anaerobically producing butanol with microorganisms. In other particular embodiments the homocetogenic microorganism of the co-culture is cultured in a fermentor until it produces a concentration of ethanol of at least 1 g/L and the butyrogenic microorganism is added to the fermentor to produce the microorganism co-culture.

In other aspects of the invention a microorganism co-culture having a nucleotide sequence defining a gene for NADPH dependent Co reductase and a nucleotide sequence defining a gene for at least one of a Butyryl-CoA acetate transferase and Butyrate kinase is provided. In particular embodiments the co-culture has a nucleotide sequence defining a gene for Butyl acetate transferase and/or a gene for Butyrate kinase.

In particular embodiments the co-culture of the invention includes C. kluyveri. In other embodiments the co-culture includes one or more homoacetogenic microorganisms selected from the group consisting of C. ljungdahlii, C. ragsdaeli, C. authoethanongenum and C. coskatii. In yet other embodiments the co-culture comprises a mixture of a homoacetogenic microorganism and a butyrogenic microorganism.

In other particular embodiments the homocetogenic microorganism of the co-culture is cultured in a fermentor until it produces a concentration of ethanol of at least 1 g/L or at least 10 g/L and the butyrogenic microorganism is added to the fermentor to produce the microorganism co-culture.

In other aspects of the invention a syntrophic co-culture of anaerobic microorganisms for producing butanol from CO or CO₂ and H₂. In particular embodiments the co-culture of microorganisms contains at least one microorganism having at least one nucleotide sequence that encodes a gene to produce an NADPH dependent CoA reductase (NADPH CoAR) and at least one additional microorganism that encodes a gene for producing a Butyryl-CoA acetate transferase (BuCoAAT) or a Butyrate kinase (BuK) is provided. The co-culture is exposed to gaseous substrates selected from the group consisting of carbon monoxide, carbon dioxide and hydrogen or combinations thereof so that a C1-fixing microorganism containing an NADPH CoAR gene and a C4-producing microorganism containing at least one of the BuCoAAT or BuK gene under conditions effective for the co-culture to convert the gaseous substrate into butanol or/and into butyric acid so that the microorganism composition of the present invention can produce butanol. In most cases the gaseous substrate is syngas and the C4-producing microorganism is a butyrogen.

### BRIEF DESCRIPTION OF THE DRAWINGS

These and other objects, features, and embodiments of the invention will be better understood from the following detailed description taken in conjunction with the drawings, wherein:

FIG. 1 is a diagram of a schematic conversion path showing the production of n-butanol from a substrate input of syngas.

FIG. 2 is a detailed diagram of the BuCoAAT pathway showing the conversion of acetate and ethanol conversion by a butyrogen to produce butyrate.

FIG. 3 is a detailed diagram of the BuK pathway showing the conversion by a butyrogen to produce butyrate.

FIG. 4 is a detailed diagram of the Wood-Ljundahl and Acetyl CoA conversion pathway showing the conversion of syngas by a homoacetogen to produce ethanol and acetate.

FIG. 5(a) is a PCR screen using probes targeted to an NADPH CoAR (NADPH dependent CoA reductase) and a BuCoAAT(butyryl-CoA acetate transferase) for analysis of a syntrophic co-culture that includes C. autoethanogenum and a consortia of at least two butanol producing microorganisms.

FIG. 5(b) is a PCR screen using probes targeted to an NADPH CoAR (NADPH dependent CoA reductase) and a BuCoAAT(butyryl-CoA acetate transferase) for analysis of a syntrophic co-culture that includes C. ragsdalei, C. coskatii, and a butyrogenic consortia of microorganisms.

FIG. 6(a) is a PCR screen using probes targeted to an NADPH CoAR (NADPH dependent CoA reductase) and BuK (butyrate kinase genes) for analysis of a syntrophic co-culture that includes C. autoethanogenum and a consortia of two butanol producing microorganisms.

FIG. 6(b) is a PCR screen using probes targeted to an NADPH CoAR (NADPH dependent CoA reductase) and aBuK (butyrate kinase genes) for analysis of a syntrophic co-culture that includes C. ragsdalei, C. coskatii, and a butyrogenic consortia of microorganisms.

FIGS. 7(a)-7(c) show sequence IDs for three butyrate production genes identified in C. carboxidivorans and C. kluyveri.

FIG. 7 (a) shows a DNA sequence alignment of the BuCoAAT gene from C. carboxidivorans and the first gene from C. kluyveri. FIG. 7 (b) provides the DNA sequence of the BuCoAAT gene from C. carboxidivorans. FIG. 7 (c) shows a first DNA and a second DNA sequence of the Bu CoAAT from C. kluyveri.

FIG. 8a shows butyrate production gene sequences identified in C. carboxydivorans for three BuK genes. FIG. 8(b) provides an alignment of two C. carboxidivorans BuK genes. FIG. 8 (c) provides an alignment of two C. carboxidivorans BuK genes (Seq ID No. 3 and Seq ID No. 9).

FIGS. 9(a)-(b) show gene sequences of the NADPH CoAR genes from four Clostridial homoacetogens.

FIG. 9 (a) showing the sequence alignment and strong homology of the four NADPH CoAR (NADPH dependent CoA reductase) gene sequences and FIG. 9(b) showing raw NADPH CoAR (NADPH dependent CoA reductase) sequences of the four homoacetogens.

FIG. 10 is a time plot of the butanol, acetate, butyrate and ethanol production from a 2 liter fermentation run using the discovered co-culture of microorganisms.

FIG. 11 is a time plot of butanol and ethanol production and hydraulic retention time (HRT) from a 10,000 gallon fermentor using the discovered co-culture of this invention.

FIG. 12 is an alignment of BCoATT C. kluyveri(Ck) and C. carboxidivorans (Cc) over 145 bp probe region.

FIG. 13 is an alignment of BCoATT C. kluyveri and C. carboxidivorans over 101 bp probe region.

FIG. 14 is an alignment of Seq. ID No. 4 C. carboxidivorans with Seq. No. 3 C. carboxidivorans over a 180 bp probe region

FIG. 15 is an alignment of C. carboxidivorans BuK-1 (Seq. ID No. 3) with C. difficile (Seq. ID No. 5) over entire gene.

### DETAILED DESCRIPTION OF THE INVENTION

The invention provides a syntrophic co-culture of microorganisms for the production of butanol and other C4-containing products from syngas.

As used herein, synthesis gas (syngas) is a gas containing carbon monoxide, carbon dioxide and frequently hydrogen. “Syngas” includes streams that contain carbon dioxide in combination with hydrogen and that may include little or no carbon monoxide. “Syngas” may also include carbon monoxide gas streams that may have little or no hydrogen.

As used herein, the term “syntrophic” refers to the association of two or more different types (e.g. organisms, populations, strains, species, genera, families, etc.) of anaerobic microorganisms which are capable of forming a tightly associated metabolic relationship.

As used herein, the term “co-culture” of microorganisms refers to joint incubation or incubation together, of the syntrophic microorganisms. In the context of the present invention, the co-culture does not require cellular population growth during the joint incubation of the syntrophic microorganisms.

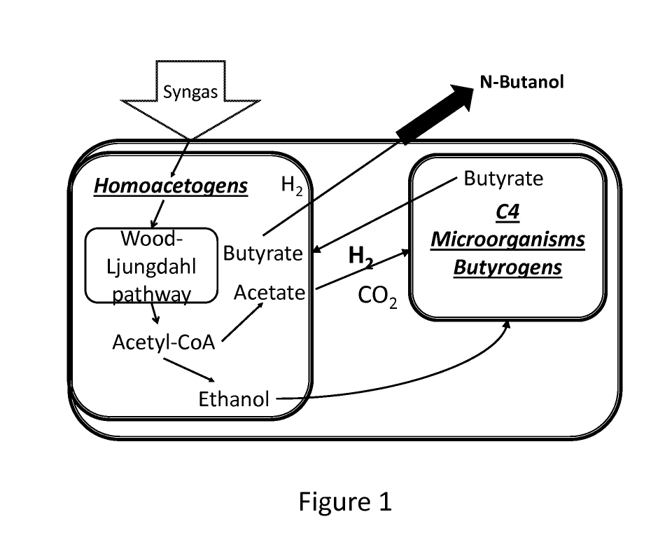

In one embodiment of the invention illustrated in FIG. 1, two types of anaerobic microorganism are utilized to create the syntrophic co-cultures for production of butyrate and butanol. The first type of microorganism in the syntrophic co-culture is a primary C1-fixing homacetogenic microorganism, which utilizes syngas as the sole carbon and electron source and produces C1 compounds such as ethanol and acetate as the dissimilatory metabolite products. The second type of microorganism in the syntrophic co-culture is capable of growing on the dissimilatory metabolites of the C1-fixing homacetogenic microorganism (ethanol and acetate) as its sole carbon and/or electron source to produce a C4-carbon molecule, such as butanol or butyric acid, as its primary product or together with syngas (as additional carbon and/or electron source) convert the metabolites of the C1-carbon fixing microorganism to C4-carbon molecules. This second microorganism shall be referred to herein as the C4-butyrate producing microorganism. Advantageously, the C1-fixing homacetogenic microorganism may also be capable of converting the butyrate produced by the C4-producing microorganism into butanol and more often n-butanol. The term “butanol” refers to all four isomers of C4 alcohol (e.g. 2-butanol, isobutanol, 1-butanol and tert-butanol) and the term “n-butanol” refers to 1-butanol.

The C1-fixing microorganisms of the invention are also homoacetogens. Homoacetogens have the ability, under anaerobic conditions, to produce acetic acid and ethanol from the substrates, CO+H₂O, or H₂+CO₂ or CO+H₂+CO₂. The CO or CO₂ provide the carbon source and the H₂ or CO provide the electron source for the reactions producing acetic acid and ethanol.

The homoacetogen organism typically has the primary Wood Ljungdahl pathway to convert the CO and H₂/CO₂ from the syngas feed to ethanol and acetate which are then utilized by the butyrogens to produce butyrate. The homoacetogens can uptake the butyrate and very efficiently convert it to n-butanol because of favored thermodynamics. Such symbiosis if preferably developed to form a very close association between the C₁ fixing and the C₄ producing microorganisms so that interspecies proton and electron transfer occur very efficiently across very short distances (approximately 1 micron). Such conditions achieve very good product concentrations (8-10 g/liter n-butanol) and yields (˜80% of electrons to n-butanol) in a single fermenter system. This combination of microorganism co-culture and substrates vastly improves the n-butanol production over that produced by a single culture fermentations. This discovery enables high yield production of butanol directly from syngas and leads to economical and efficient production processes for butanol from a wide range of feedstocks.

C1-fixing microorganisms suitable for use in the inventive method include, without limitation, homoacetogens such as Clostridium ljungdahlii, Clostridium autoethanogenum, Clostridium ragsdalei, and Clostridium coskatii. Additional C1 fixing microorganisms that are suitable for the invention include Alkalibaculum bacchi, Clostridium thermoaceticum, and Clostridium aceticum.

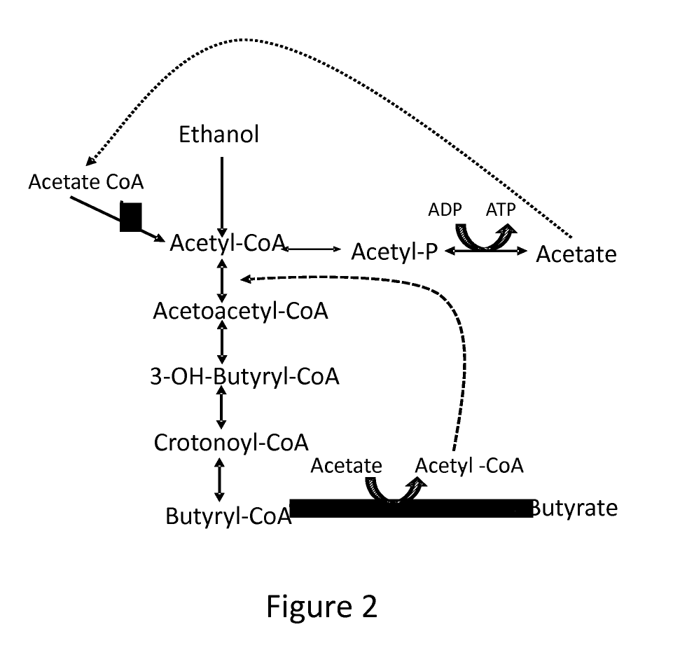

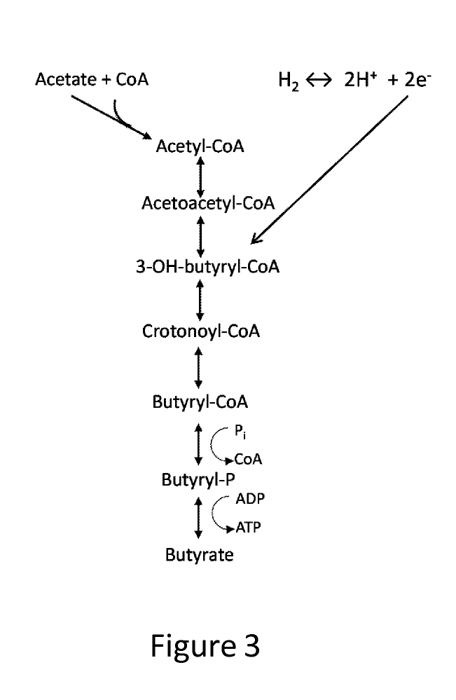

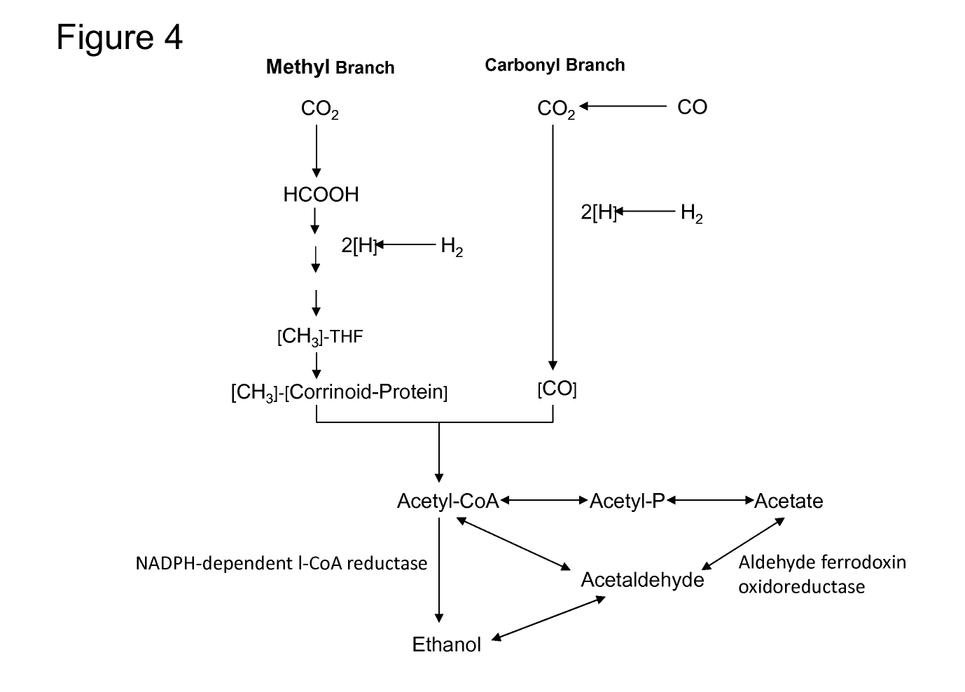

In particular embodiments syntrophic C4-producing microorganisms are a butyrogen capable of growing on ethanol and/or acetate as their primary carbon source. Butyrogens refers to any microorganism capable of converting syngas intermediates, such as ethanol and acetate, and some hydrogen to primarily n-butyrate. Butyrogens of the invention utilize at least one of two distinct pathways for butyrate production—the Butyrl CoA Acetate Transferase pathway (shown in FIG. 2) and the Butyrl Kinase (BuK) pathway (shown in FIG. 3). As can be seen from the FIGS. 2 and 3, the Butyryl CoA Acetyl Transferase (BuCoAT) pathway converts ethanol and acetate to butyrate:

Ethanol+Acetate [symbol] Butyrate+H₂O

As shown in FIG. 3 the BuK pathway converts acetate and hydrogen to Butyrate.

2H₂+2Acetate [symbol] Butyrate+2H₂O

In the BuCOAT pathway ethanol and acetate are converted to butyrate through a Butyrl CoA intermediate. Similarly acetate plus reducing equivalents through H₂ oxidation are converted to butyrate through a butryl CoA intermediate. The pathways differ in their conversion steps from butyryl CoA to butyrate. The BuCoAT pathway converts butyrl CoA to butyrate through the BuCoAT enzyme while transferring the CoA moiety to acetate to form acetyl-CoA, which can later be used to form more butyrate. At the same time the BuK pathway converts butyryl CoA through a phosphotransbutyrylase and BuK enzyme. The NADPH-dependent CoA reductase converts butyryl-CoA directly into butanol in a 4 electron transfer reaction using NADPH. Suitable butyrigens for this invention include any microorganism that contains either or both of the BuCoAt pathway and BuK pathway and can grow on acetate and ethanol or on acetate and hydrogen as typically found in syngas.

While many microorganism are known to produce butyrate from various carbohydrate sources (C. butyricum, C. acetobutylicum, C. tyrobutyricum, C. beijerinckii, C. pasteurianum, C. barkeri, C. thermobutyricum, C. thermopalmarium, Butyrvibrio, Sarcina, Eubacterium, Fusobacterium, and Megasphera), only a few are known to grow exclusively on ethanol, acetate or syngas such as Clostridium kluyveri, Clostridium carboxidivorans, and Butyribacterium methylotrophicum.

This invention can employ as the syntrophic co-culture a combination of microorganisms that provides unique and identifiable combination of genes that are not present in organisms that can directly ferment syngas to butanol or in other butyrogens that can utilize ethanol or acetate together with hydrogen to product butyrate.

The pairing of the homoacetogens with the butyrogens provided herein demonstrates a vast improvement over the prior art. Table 1 shows a comparison of single culture production to the use of syntophic cultures. As the results show, a four-fold increase in the concentration of n-butonal was achieved. Thus, in particular embodiments of the invention high yield production of butanol directly from syngas was achieved which leads to economical and efficient production processes for butanol from a wide range of feedstocks.

TABLE 1

| | n-Butanol | n-Butanol | | |
|---|---|---|---|---|
| | concen- | yield | Ethanol by | |
| | tration | achieved | product | |
| Bio-conversion | achieved | (% of | (% of | |
| method | (g/l) | electrons) | electrons) | Reference |
| B. | 2 to 3 | 40 to 45% | 10-20% | See Grethlein et |
| methylotrophicum | | | | al., Journal of |
| (single culture) | | | | Fermentation and |
| | | | | Bioengineering, |
| | | | | 72, 1, 58-60 |
| | | | | (1991). |
| C. | 2 to 2.5 | 20 to 25% | 20 to 25% | Liou et al. |
| carboxidivorans | | | | International |
| (single culture) | | | | Journal of |
| | | | | Systematic and |
| | | | | Evolutionary |
| | | | | Microbiology |
| | | | | 55(5): 2085-2091) |
| | | | | (2005) |
| | | | | Guillaume et al. |
| | | | | PLoS One, 5(9)) |
| | | | | (2010) |
| Syntrophic | 8 to 9 | 60 to 80% | 10 to 25% | Examples 1 and 2 |
| Co-culture | | | | |

The present invention provides a combination of the genes for an NADPH dependent CoA reductase and for the genes of a Butyryl-CoA acetate transferase and/or a Butyrate kinase such that this unique gene combination can make butanol from one or more syngas components. NADPH dependent CoA reductase does not occur in the heteroacetogenic organisms nor do the Butyryl-CoA Acetate transferase or Butyrate kinase occur in the homocetogenic organism. The genetic novelty of these genes was established by identifying key genes in the syntrophic butyrate production pathway using targeted gene probes. The novelty of the butyryl-CoA transferase genes in the butanologenic consortia appears to be a highly specific transferase reaction. Hence, unique combinations of genes exist in these syntrophic co-cultures that do not occur in other organisms that have been used to produce butanol.

It was also surprisingly found that this combination of genes existed no matter which homoacetogens or heteroacetogens were used and that the combination of genes, present in the syntrophic combination of the organisms containing these genes, will stay in close association without either of the organisms washing out from the co-culture in a fermentation. Most advantageously this unique combination of genes produce butanol from syngas at high titers that were unachievable with other microorganisms absent the use of multiple substrates. (See Table 1.)

A successful syntrophic relationship between the different microorganisms of the present invention require that the homoacetogens and the butyrogens are brought into close physical association with each other. In particular embodiments the C1 converting homoacetogens with the Wood Ljundahl pathway and the NADPH-dependent CoA reductase genes are brought together in an intimately mixed co-culture with the butyrogens having the BuCoAAT or the BuK genes. In another embodiment of the invention the C1 converting homoacetogens will have an NADPH CoAR (NADPH dependent CoA reductase) gene to further increase the production of butanol.

In one method of the invention, the syntrophic co-culture is formed by first growing a single species or a combination of known homoacetogen species on a syngas feed. Growth of the homoacetogens continues until they produce ethanol and acetate, normally at a concentration of at least 1 g/1 and more typically in a moderate concentration range of 8 to 15 g/1 and preferably at a concentration of 10 g/1 and a cell concentration producing an optical density (O.D.) of about 2.0. Once the homoacetogens have produced a desired concentration of ethanol and acetate and the fermenter has reached a desired O.D., the homoacetogens are inoculated with one or more selected butyrogen species that are enriched from growth on acetate, ethanol and syngas. By maintaining growth and operating conditions such as pH, dilution rate, key nutrients etc., a stable syntrophic co-culture is developed that forms very close associations between the different microorganisms.

Those skilled in the art will be aware of other methods to initiate and grow the co-culture. Such methods may include the use of different substrates to first grow the butyrogen and then inoculate the fermentation medium containing the butyrogen with the homoacetogen. Another method for establishing a syntrophic association capable of converting syngas to butanol involves the growing of two or more defined cultures and establishing the pairing of these separate cultures.

Another method of pairing involves first growing the C4-producing butyrogen in a fermenter using ethanol and acetate as substrates until maximum productivity targets of butyric acid have been reached. Once the maximum productivity target has been reached a seed culture of the C1-fixing homoacetogen is added directly to the fermenter containing the butyrogen culture. Syngas mass transfer to the fermentation vessel is gradually increased to balance the gas consumption of the C1-fixing homoacetogen. The ethanol or acetate used to grow the butyrogen are gradually decreased to zero as the C1-fixing homoacetogen begins to provide this substrate.

A modification of this last method of establishing a syntrophic culture involves first growing the C4-producing butyrogen culture in a fermenter with a biofilm support material that is either stationary or floating within the reactor. An example of such material is the Mutag Biochips. This method allows the butyrogen microorganism to first establish a biofilm on the carrier material thereby increasing the cell retention time versus the HRT of the fermenter. Again, target butyrogen productivity is reached before seeding the fermenter with the C1-fixing homoacetogen.

Another method to establish a syntrophic culture capable of producing butanol from syngas involves the initial mixing together of two or more cultures, one of which is a C1-fixing homoacetogen capable of growing on syngas and producing ethanol and acetate. The other culture(s) is a C4-producing butyrogen capable of converting ethanol or acetate to butyrate. Ethanol and acetate feed can gradually be decreased to zero as the production of these substrates by the C1-fixing homoacetogens increases to balance the substrate needs of the butyrogen production.

Suitable pairings of microorganisms for the syntrophic co-culture composition of this invention are identified by the presence of key genes in the syntrophic pathways for the homoacetogenic and butyrogenic microorganism. These pathway are typically identified by using targeted gene probes. The probes are targeted toward identifying the presence of genes in the syntrophic consortium that encode for an NADPH CoAR gene, at least one BuCoAAT gene or one BuK gene. The presence or absence of these genes can be further determined using genomic DNA and suitable probes. Further description of the gene sequences are provided in the Examples.

The methods of the present invention can be performed in any of several types of fermentation apparatuses that are known to those of skill in the art, with or without additional modifications, or in other styles of fermentation equipment that are currently under development. Examples include but are not limited to conventional stirred tank fermenters (CSTRS), bubble column bioreactors (BCBR), membrane supported bioreactors (MSBR), two stage bioreactors, trickle bed reactors, membrane reactors, packed bed reactors containing immobilized cells, etc. Bioreactors may also include a column fermenter with immobilized or suspended cells, a continuous flow type reactor, a high pressure reactor, or a suspended cell reactor with cell recycle. Furthermore, reactors may be arranged in a series and/or parallel reactor system which contains any of the above-mentioned reactors. For example, multiple reactors can be useful for growing cells under one set of conditions and generating n-butanol (or other products) with minimal growth under another set of conditions.

Establishing the necessary close association of the co-culture may be influenced by the type of bioreactor employed for practice of the invention. For example in the case of planktonic type bioreactors the syntrophic co-culture may continue in a growth phase and be passaged up to larger fermentation vessels. In the case of an MSBR, an established co-culture from a planktonic fermenter may be used to inoculate the membranes. However, an MSBR may also be inoculated by a series of inoculations that alternate between addition of the homoacetogen and addition of the butyrogen.

These apparatuses will be used to develop and maintain the C1-fixing homoacetogen and butyrogen cultures used to establish the syntrophic metabolic association. The chief requirements of such an apparatus include:
a. Axenicity;
b. Anaerobic conditions;
c. Suitable conditions for maintenance of temperature, pressure, and pH;
d. Sufficient quantities of substrates are supplied to the culture;
e. Optimum mass transfer performance to supply the gases to the fermentation medium
e. The end products of the fermentation can be readily recovered from the bacterial broth.

Suitable gas sources of carbon and electrons are preferably added during the inoculation. In addition to those already described these gaseous sources come from a wide range of materials and include “waste” gases such as syngas, oil refinery waste gases, steel manufacturing waste gases, gases produced by steam, autothermal or combined reforming of natural gas or naphtha, biogas and products of biomass, coal or refinery residues gasification or mixtures of the latter. Sources also include gases (containing some H₂) which are produced by yeast, clostridial fermentations, and gasified cellulosic materials. Such gaseous substrates may be produced as byproducts of other processes or may be produced specifically for use in the methods of the present invention. Those of skill in the art will recognize that any source of substrate gas may be used in the practice of the present invention, so long as it is possible to provide the microorganisms of the co-culture with sufficient quantities of the substrate gases under conditions suitable for the bacterium to carry out the fermentation reactions.

In one embodiment of the invention, the source of CO, CO₂ and H₂ is syngas. Syngas for use as a substrate may be obtained, for example, as a gaseous product of coal or refinery residues gasification.

In addition to those sources as described, syngas can be produced by gasification of readily available low-cost agricultural raw materials expressly for the purpose of bacterial fermentation, thereby providing a route for indirect fermentation of biomass to alcohol. There are numerous examples of raw materials which can be converted to syngas, as most types of vegetation could be used for this purpose. Suitable raw materials include, but are not limited to, perennial grasses such as switchgrass, crop residues such as corn stover, processing wastes such as sawdust, byproducts from sugar cane harvesting (bagasse) or palm oil production, etc. Those of skill in the art are familiar with the generation of syngas from such starting materials. In general, syngas is generated in a gasifier from dried biomass primarily by pyrolysis, partial oxidation, and steam reforming, the primary products being CO, H₂ and CO₂. The terms “gasification” and “pyrolysis” refer to similar processes; both processes limit the amount of oxygen to which the biomass is exposed. The term “gasification” is sometimes used to include both gasification and pyrolysis.

Combinations of sources for substrate gases fed into the fermentation process may also be utilized to alter the concentration of components in the feed stream to the bioreactor. For example, the primary source of CO, CO₂ and H₂ may be syngas, which typically exhibits a concentration ratio of 37% CO, 35% H₂, and 18% CO₂, but the syngas may be supplemented with gas from other sources to enrich the level of CO (i.e., steel mill waste gas is enriched in CO) or H₂.

The syntrophic co-cultures of the present invention must be cultured and used under anaerobic conditions. As used herein, “anaerobic conditions” means the level of oxygen (O₂) is below 0.5 parts per million in the gas phase of the environment to which the microorganisms are exposed. One of skill in the art will be familiar with the standard anaerobic techniques for culturing these microorganisms (Balch and Wolfe (1976) Appl. Environ. Microbiol. 32:781-791; Balch et al., 1979, Microbiol. Rev. 43:260-296), which are incorporated herein by reference. Other operating conditions for the established co-culture will usually include a pH in a range of 5 to 7.

A suitable medium composition used to grow and maintain syntrophic co-cultures or separately grown cultures used for sequential fermentations, includes a defined media formulation. The standard growth medium is made from stock solutions which result in the following final composition per Liter of medium. The amounts given are in grams unless stated otherwise. Minerals: NaCl, 2; NH₄Cl, 25; KCl, 2.5; KH₂PO₄, 2.5; MgSO₄.7H₂O, 0.5; CaCl₂.2H₂O, 0.1. Trace metals: MnSO₄.H₂O, 0.01; Fe(NH₄)₂(SO₄)₂.6H2O, 0.008; CoCl₂.6H2O, 0.002; ZnSO₄.7H2O, 0.01; NiCl₂.6H2O, 0.002; Na₂MoO₄.2H₂O, 0.0002, Na₂SeO₄, 0.001, Na₂WO₄, 0.002. Vitamins (amount, mg): Pyridoxine HCl, 0.10; thiamine HCl, 0.05, riboflavin, 0.05; calcium pantothenate, 0.05; thiocticacid, 0.05; p-aminobenzoic acid, 0.05; nicotinic acid, 0.05; vitamin B12, 0.05; mercaptoethane sulfonic acid, 0.05; biotin, 0.02; folic acid, 0.02. A reducing agent mixture is added to the medium at a final concentration of 0.1 g/L of cysteine (free base); and 0.1 Na₂S.2H₂O. Medium compositions can also be provided by yeast extract or corn steep liquor or supplemented with such liquids.

In general, fermentation of the syntrophic co-culture will be allowed to proceed until a desired level of butanol is produced in the culture media. Preferably, the level of butanol produced is in the range of 2 grams/liters to 75 grams/liters and most preferably in the range of 6 grams/liter to 15 grams/liter. Alternatively, production may be halted when a certain rate of production is achieved, e.g. when the rate of production of a desired product has declined due to, for example, build-up of bacterial waste products, reduction in substrate availability, feedback inhibition by products, reduction in the number of viable bacteria, or for any of several other reasons known to those of skill in the art. In addition, continuous culture techniques exist which allow the continual replenishment of fresh culture medium with concurrent removal of used medium, including any liquid products therein (i.e. the chemostat mode). Also techniques of cell recycle may be employed to control the cell density and hence the volumetric productivity of the fermentor.

The products that are produced by the microorganisms of this invention can be removed from the culture and purified by any of several methods that are known to those of skill in the art. For example, butanol can be removed by distillation at atmospheric pressure or under vacuum, by adsorption or by other membrane based separations processes such as pervaporation, vapor permeation and the like.

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined to provide additional guidance to the practitioner regarding the description of the invention.

### EXAMPLES

The Examples which follow are illustrative of specific embodiments of the invention, and various uses thereof. They are set forth for explanatory purposes only, and are not to be taken as limiting the invention.

### Example 1

### Establishment of Stable Syntrophic Pairing of a Homoacetogen with Butyrogens

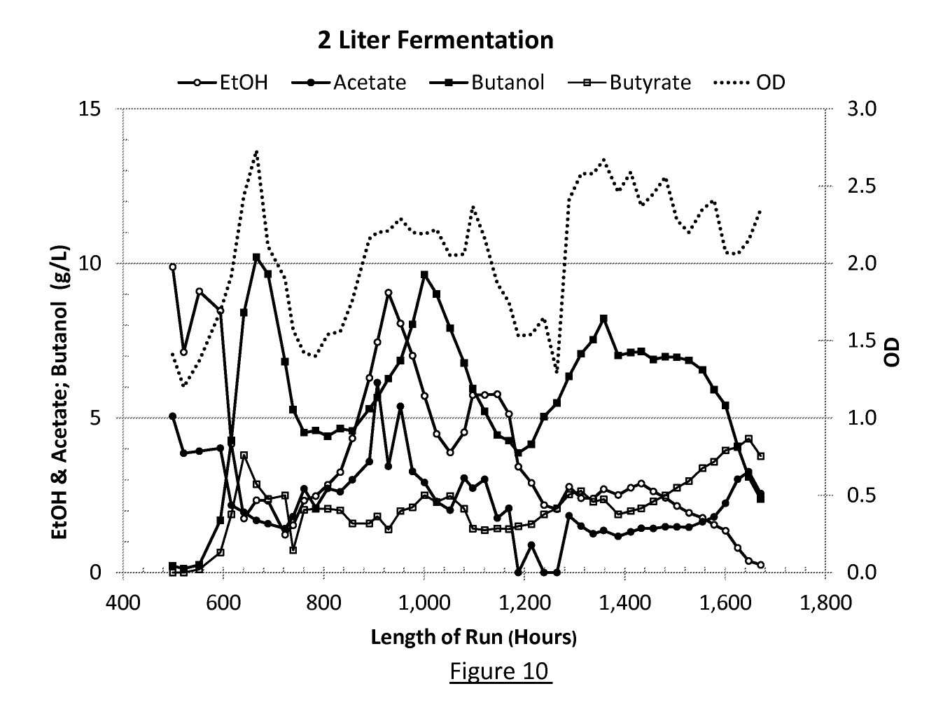

A 2-liter fermentation experiment was run in order to establish a syntrophic pairing of a type strain homoacetogen, Clostridium autoethanogenum, and a mixed culture of two butyrogens known to produce butyrate and have at least one gene for BuCoAAT and one gene for BuK. The mixed culture of Clostridium autoethanogenum was first grown to an O.D. of 1.7 on minimal media and syngas with a composition of H₂-56%, CO-22%, CO₂-5%, and CH₄-17% (mol %), 60 mL/min. gas flow rate and agitation between 500-600 rpm. The ethanol and acetate concentrations were at 10 and 5 g/L respectively prior to the addition of 200 mL of the mixed butyrogen culture. FIG. 10 shows the concentration of the ethanol, acetate, butyrate and butanol in the fermenter at the time of mixed butyrogen culture addition. The butyrate and butanol concentrations slowly increased and 6 days after inoculation with the butyrogens, butanol and butyrate concentration of 8.4 and 3.8 g/L, respectively were achieved. The increase in butanol and butyrate coincided with a decrease in ethanol and acetate to concentrations of 1.8 and 2.0, respectively. During this time period, more than 70% of the electrons consumed as syngas were being converted to butanol and butyrate.

### Example 2

### Establishment of Stable Syntrophic Pairing of Two Homoacetogens with Butyrogenic Consortia

A fermentation experiment, similar to that of Example 1, was run. The main difference was that the syntrophic co-culture used two homoacetogens, Clostridium ragsdalii and Clostridium coskatii, in combination with an enriched consortium of butyrogens known to produce butyrate and having at least one of the genes for BuCoAAT and one of the genes for BuK. All of the conditions were the same as in Example 1 including the addition of butyrogen to the fermenter after establishing the homocetogens in the fermenters. The fermentation produced n-butanol by converting syngas with the syntrophic co-culture that included a suspended culture of the consortium.

### Example 3

### Molecular Detection of NADPH-Dependent CoA Reductase and BuCoAAT Genes in Butanol-Producing Consortia

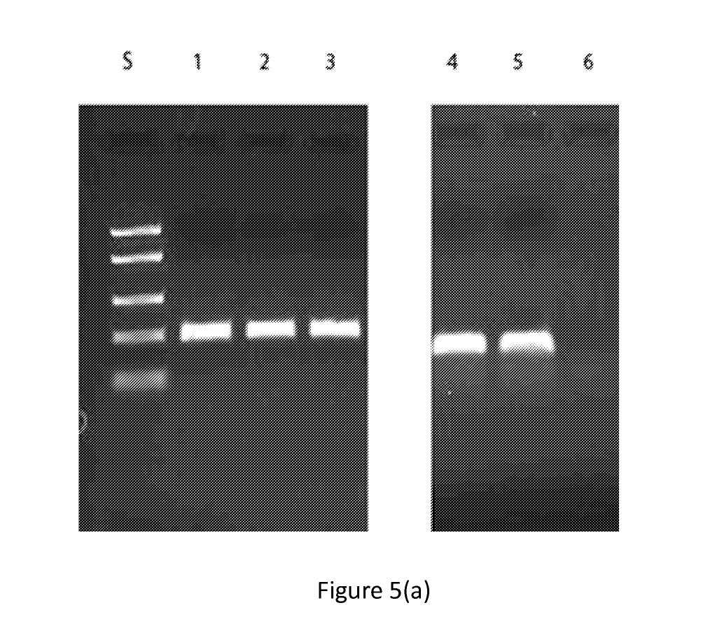

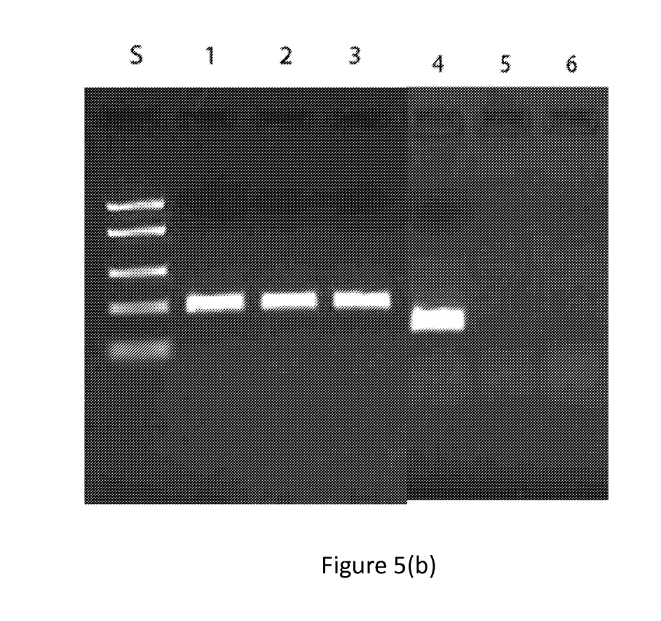

High butanol-producing consortia were screened for the presence of key genetic targets using molecular probes. The PCR probes were designed to detect the presence of NADPH CoAR and BuCoAAT genes. The primer sequences for the NADPH CoAR gene were obtained from sequence alignments of the genes from four homoacetogen sequences. The forward and reverse primers used were: Forward, 5′-AAGCGGTGATACTTTACCAA-3′(SEQ ID NO. 26) and reverse 5′-GGGCCTTTTCAATATTTTCT-3′ (SEQ ID NO. 27). The primers for amplifying the Butyryl-CoA acetate transferase gene(s) in butyrogens were obtained from a sequence alignment of the Clostridium kluyveri BuCoATT genes. The primer sequences are: forward 5′-AAAAAGGATYTDGGKATWCATTC-3′ (SEQ ID NO. 28) and reverse 5′-TCATAHARYYTYTTWGTWCCCAT-3′(SEQ ID NO. 29). Degeneracies were added to capture a broad range of butyrogens for quantitative studies. FIG. 5(a) shows the results of PCR using genomic DNA taken from samples containing strain C. autoethanogenum and two butyrogenic consortia. Both consortia samples and the pure C. autoethanogenum DNA gave a PCR product of about 200 bp using probes targeted to the NADPH CoAR genes with lanes 1 and 2 showing the PCR result for two syntrophic co-cultures and lane 3 showing the result for a pure sample of C. autoethanogenum. Additionally, PCR cycling conditions consisted of 3 minutes at 94° C. for template DNA denaturation followed by 30 cycles of 1 min. at 94° C., 30 sec. at 59° C., and 30 sec. at 72° C. All reaction mixes contained a 2×PCR dreamTaq master mix from Fermentas and the appropriate DNA template and primers at 50 nM final concentration.

FIG. 5(a), lanes 4-6, show the gel results of PCR using the same two consortia and pure C. autoethanogenum DNA as shown in FIG. 5(a) but using a probe targeting the BCoATT gene(s). Reactions were performed as described above. The butyrogenic consortia showed a product of about 150 bp using probes targeted to butyryl-CoA acetate transferase genes while C. autoethanogeum (lane 6) showed no PCR product for the BuCoAAT gene.

In FIG. 5(b), lanes 1-3, the gel results are shown for PCR that was performed with the same DNA extracted from cultures in another reactor containing the DNA for pure C. ragsdalei and C. coskatii and the butyrogenic consortia. Reactions were performed as described above for the use of the NADPH CoAR probe. The consortium DNA yielded an amplicon of about 200 bp using the NADPH CoAR probe, indicating that the homoacetogenic Clostridia genetic targets were present in the consortia sample taken from the butyrogenic reactor. The pure C. ragsdalei and C. coskatii DNA also gave an amplicon of the expected size (FIG. 5b).

In FIG. 5(b), lanes 4-6, the gel results are shown for PCR that used the same DNA extracted from the reactor containing the two homoacetogens, C. ragsdalei and C. coskatii, along with a butyrogenic consortium. Reactions were performed as described above for the use of the NADPH CoAR probe (FIG. 5(a) lanes 1-3.) The only difference in this case was the use of the butyryl-CoA acetate transferase probe. As the gel results show the butyryl-CoA acetate transferase probe only generated an amplicon with the consortia DNA and not with the pure C. ragsdalei and C. coskatii DNA, indicating that the homoacetogenic Clostridia in the butyrogenic reactor did not have BuCoAAT genes but the amplicon was solely due to the presence of butyrogenic organisms.

### Example 4

### Molecular Detection of NADPH CoAR and BuK Genes in Butanol-Producing Consortia

Butyrogenic consortia by themselves do not make butanol without the NADPH CoAR genes but can make butyrate using the butyrate kinase pathway. The butyrate can then be converted to butanol by the acetyl-CoA reductase activity found in C. autoethanogenum and the other homoacetogens.

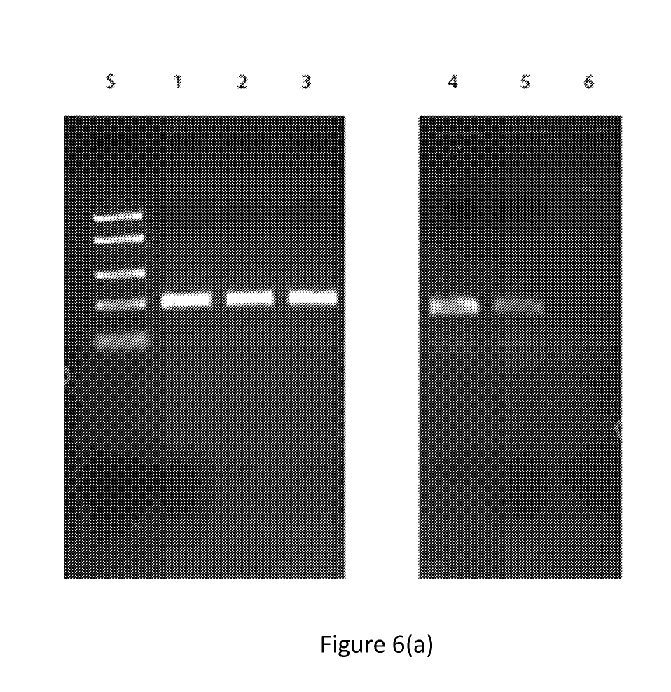

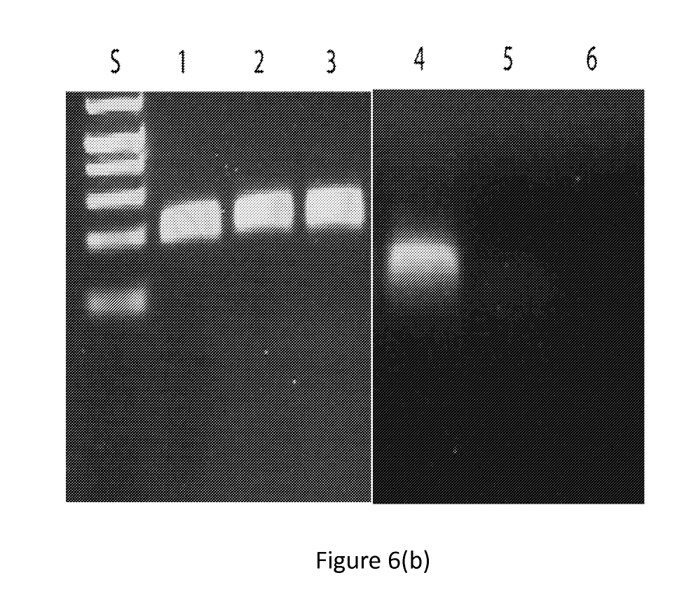

In FIG. 6(a), lanes 1-3, the gel results of PCR that was performed with DNA extracted from cultures containing the DNA from C. autoethanogenum and two consortia samples of butyrogens. Reactions were performed as described above for the use of the NADPH CoAR probe. Consortia samples amplified NADPH CoAR genes indicating that in these consortia samples acetyl-butyryl-CoA reductase genes were present and were contributing to butanol production.

In FIG. 6(a), lanes 4-6, the gel results are shown for PCR that was performed with DNA extracted from cultures containing the DNA from C. autoethnanogenum and two consortia samples of butyrogens. A PCR probe was designed to specifically amplify butyrate kinase genes in a wide variety of butyrogens and tested with consortia samples. The primers used were obtained from sequence alignments of C. carboxidivorans genes. The forward primer was 5′-AAAGAGCTGGAAAAGTTCCT-3′ (SEQ ID NO. 30) and the reverse 5′-CAAGCTTTGCTTTTTCATCT-3′ (SEQ ID NO. 31). Reactions were performed as described above for the use of the NADPH CoAR probe (FIG. 5(a) lanes 1-3.) The only difference in this case was the use of the BuK probe. Both consortia gave amplicons of about 180 bp, consistent with amplicons observed in control DNA. The results indicate that in both of these consortia samples the butyrate kinase and NADPH dependent CoA reductase gene (FIG. 6(a) lanes 1-3) were present.

In FIG. 6(b), lanes 1-3, the gel results of PCR that was performed with the DNA of pure C. ragsdalei and C. coskatii and the butyrogenic consortia. Reactions were performed as described above for the use of the NADPH CoAR probe in Example 3A. As expected, the consortium DNA yielded an amplicon of about 200 bp using the NADPH CoAR probe, indicating that the homoacetogenic Clostridia organisms were present in the consortium sample taken from the butyrogenic reactor.

In FIG. 6(b), lanes 4-6, the gel results are shown for PCR that was performed with DNA extracted from cultures containing the DNA from pure C. ragsdalei and C. coskatii and the butyrogenic consortia. Reactions were performed as described above for the use of the NADPH CoAR probe (FIG. 5(a) lanes 1-3.) The only difference in this case was the use of the BuK probe. The BuK probe only generated an amplicon with the co-culture DNA and not with the pure C. ragsdalei and C. coskatii DNA thereby indicating that the homoacetogenic Clostridia in the butyrogenic reactor did not have BuK genes but the amplicon was solely due to the presence of butyrogenic organisms.

### Example 5

### Clostridium carboxidivorans Contains Genes Encoding BuK and BuCoAAT

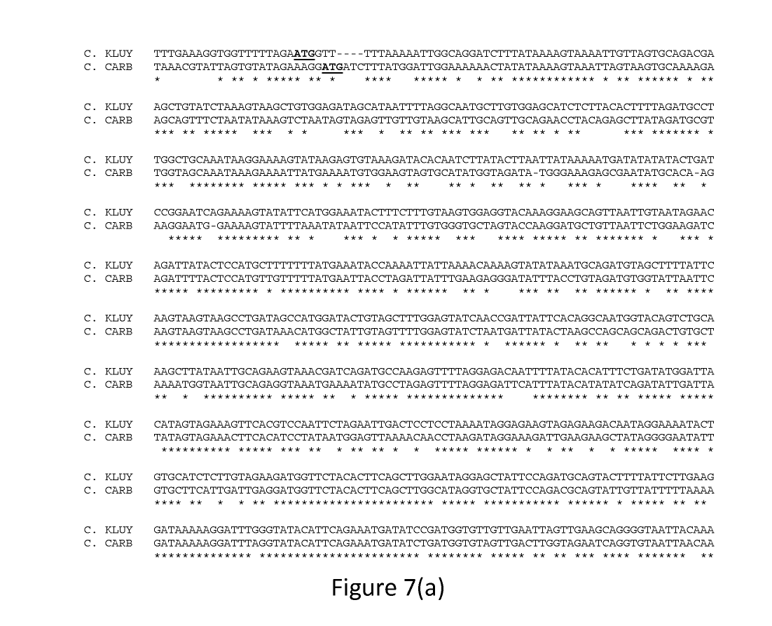

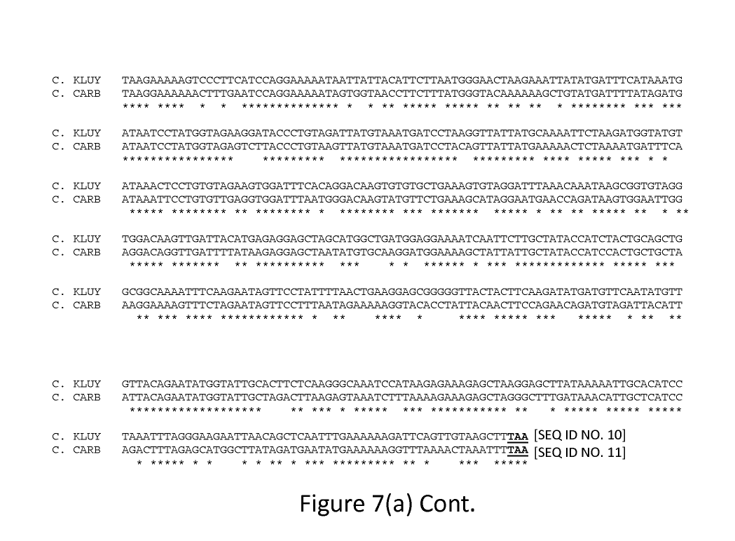

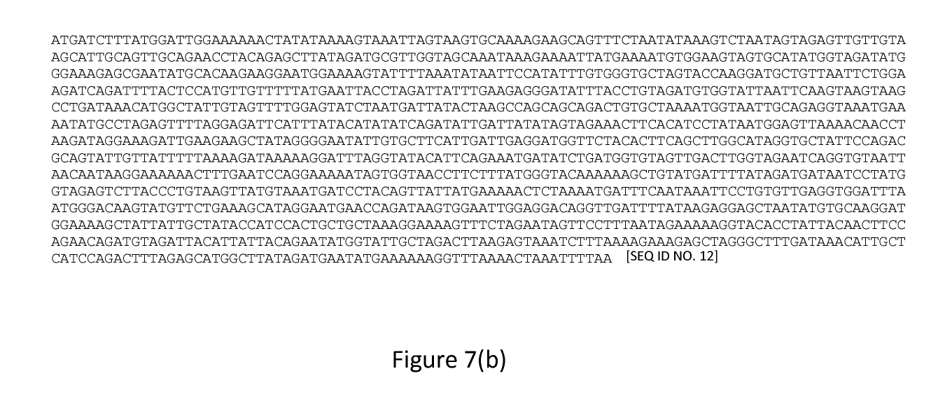

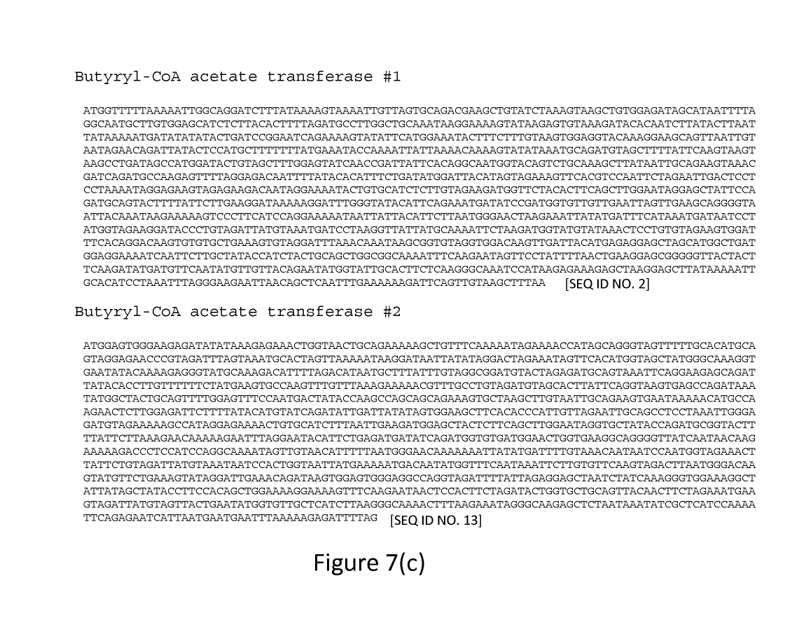

Clostridium carboxidivorans produces ethanol, acetate, butyrate and butanol when grown in the presence of syngas, which is largely a mixture of CO, H₂ and CO₂. Investigation of its genome sequence revealed two possible pathways to butyrate production with one predominating. The main route appears to be via the butyrate kinase pathway since there are three COGs annotated as such. The remaining part of this pathway is completely intact in C. carboxidivorans, that is, the phosphate transbutyrylase and all upstream genes to make butyrate and butanol are present. C. carboxidivorans also contains one gene that potentially allows the production of butyrate via the butyryl-CoA acetate transferase pathway gene and shows high homology to the genes from C. kluyveri (FIG. 7a). The percent identity of the entire C. kluyveri and C. carboxidivorans BCoATT genes was 74%. These genes appear to be quite novel since most butyrate transferases are involved in the conversion of 4-hydroxybutyryl-CoA and acetoacetate to acetoacetyl-CoA, which then goes through the butyrate pathway. That reaction is involved in amino acid catabolic pathways. The novelty of the butyryl-CoA Acetate transferase genes in the butanologenic consortia appears to be a highly specific transferase reaction. The sequences for the one BuCoAAT gene in C. carboxydivorans is given in FIG. 7(b) and for the two BuCoAAT genes in C. kluyveri are given in FIG. 7(c).

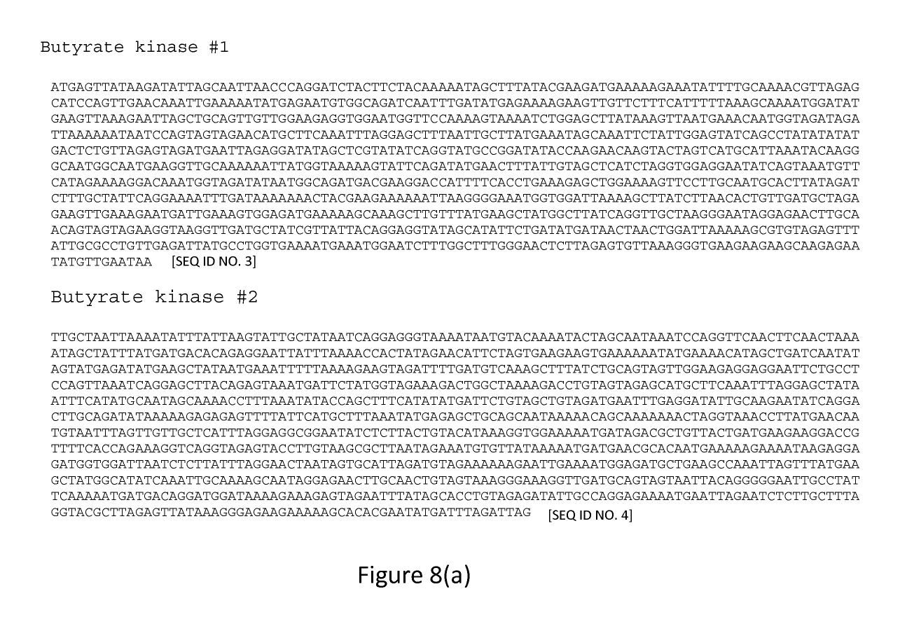

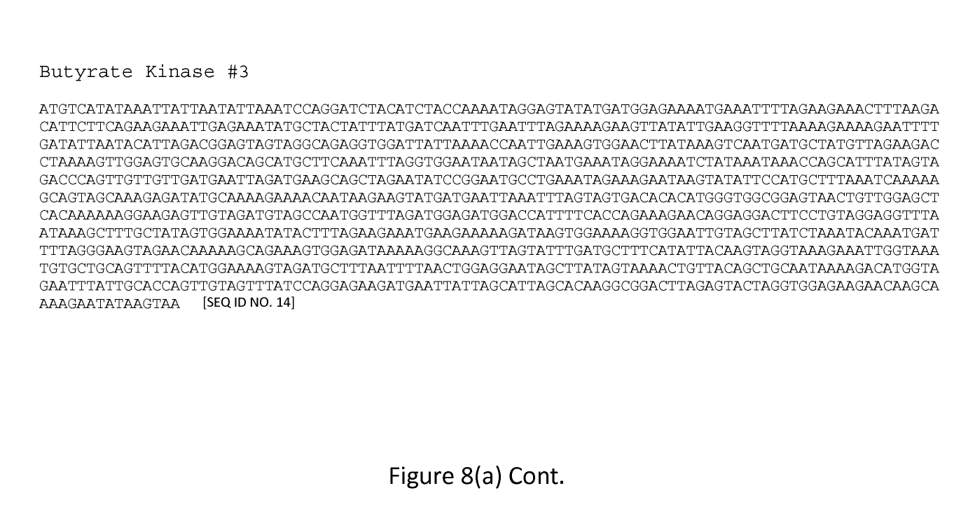

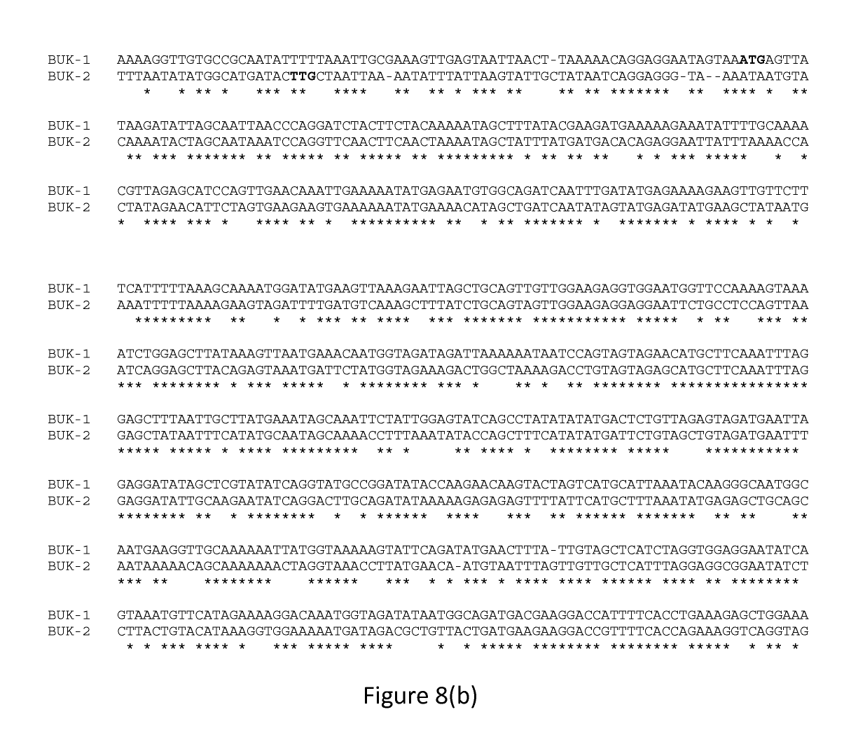

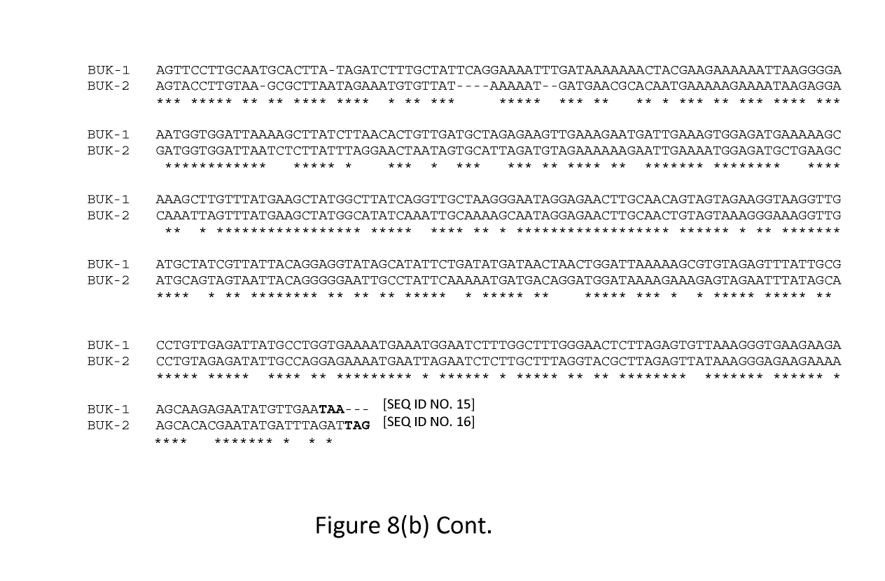

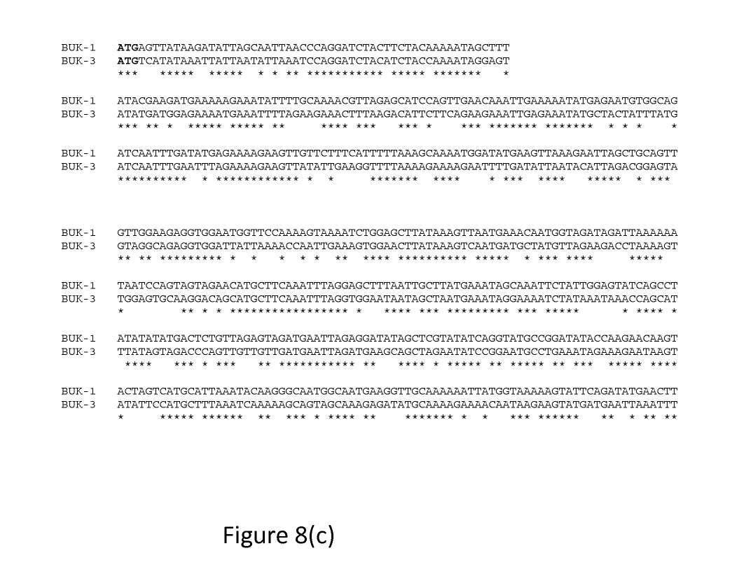

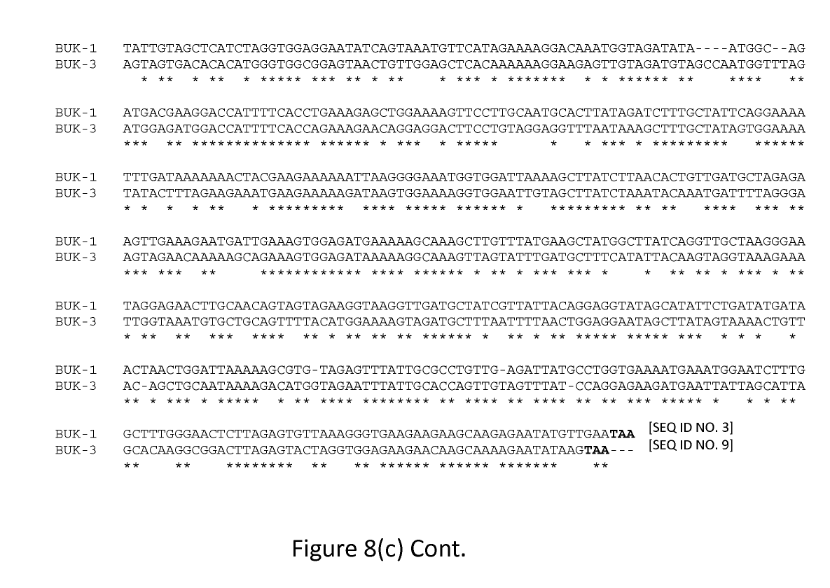

The three butyrate kinase genes identified in C. carboxidivorans are shown in FIG. (8a). When the entire C. carboxidivorans butyrate kinase (Seq. ID No. 3) was aligned pairwise with the other two butyrate kinases (FIG. 8b, 8c) the percent identities were 68% and 58%, respectively.

Interestingly, the C. carboxidivorans genome did not reveal the presence of the NADPH CoAR sequence, suggesting that these enzymes are only present in homoacetogenic Clostridia that produce ethanol from syngas. Furthermore, in contrast to the homoacetogenic Clostridia grown on syngas, C. carboxidivorans was unable to convert ketones such as acetone, butanone and pentanone to the corresponding secondary alcohols (data not shown), indicating that there is no cryptic short-chain fatty acid coenzyme A reductase activity in the cell.

### Example 6

### Clostridium kluvveri butyrate production genes

Clostridium kluyveri contains a somewhat unique metabolic niche whereby it converts ethanol and acetate to butyrate and caproate. It doesn't have the ability to convert syngas to butyrate since it lacks the Wood-Ljungdahl pathway. Examination of its genome sequence shows the presence of two butyryl-CoA acetate transferase genes and no butyrate kinases (FIG. 7(c) indicating that the production of butyrate and caproate occurs via the transferase pathway.

C. kluyveri also lacks the NADPH CoAR gene sequence and enzymatic activity that's been observed in homoacetogenic Clostridia and which is also lacking in C. carboxidivorans, a heteroacetogenic Clostridia described in Example 5.

### Example 7

### Homoacetogenic Clostridia Containing NADPH CoAR Sequences

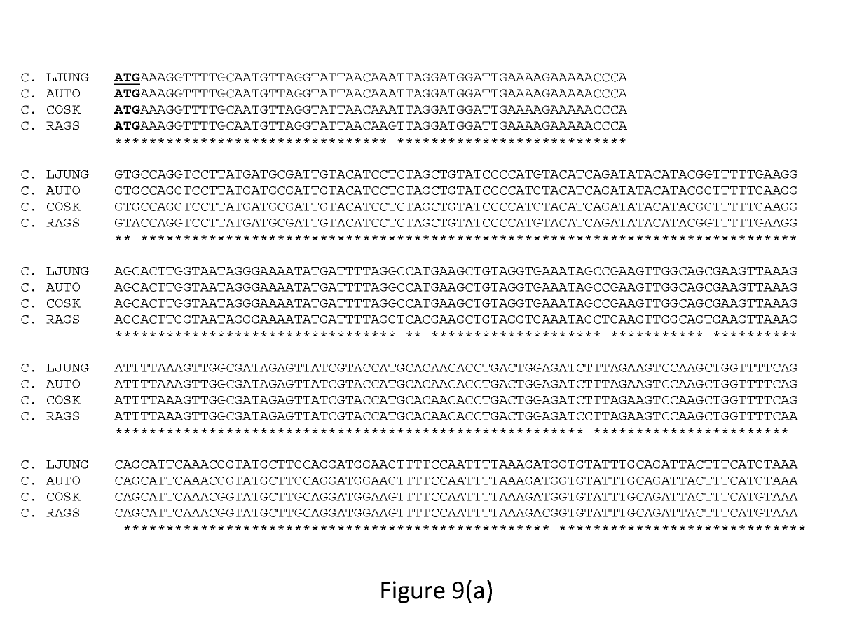

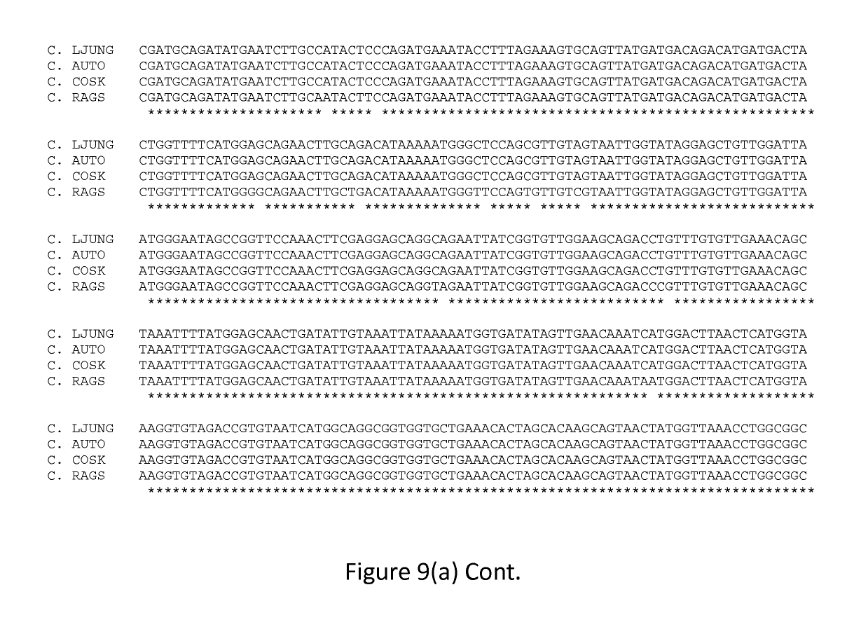

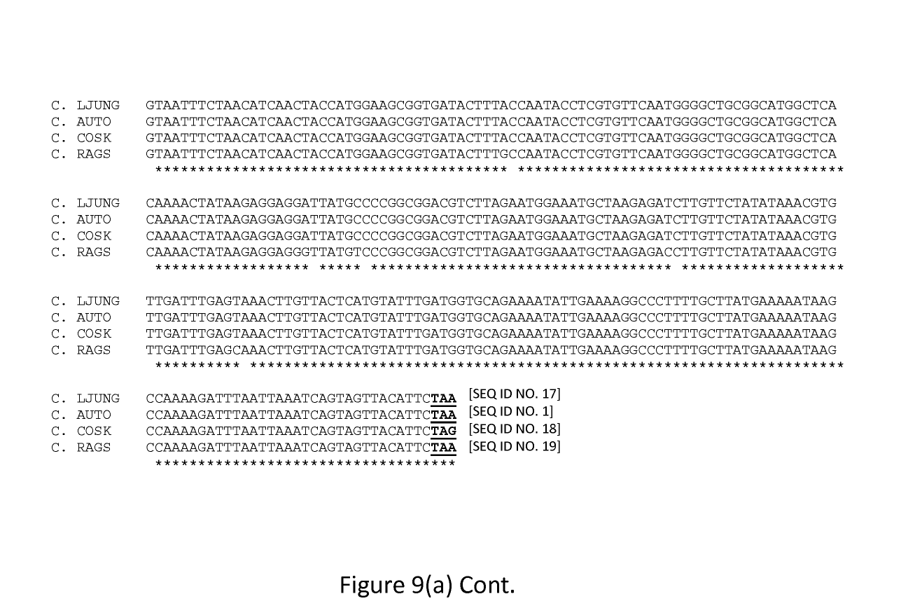

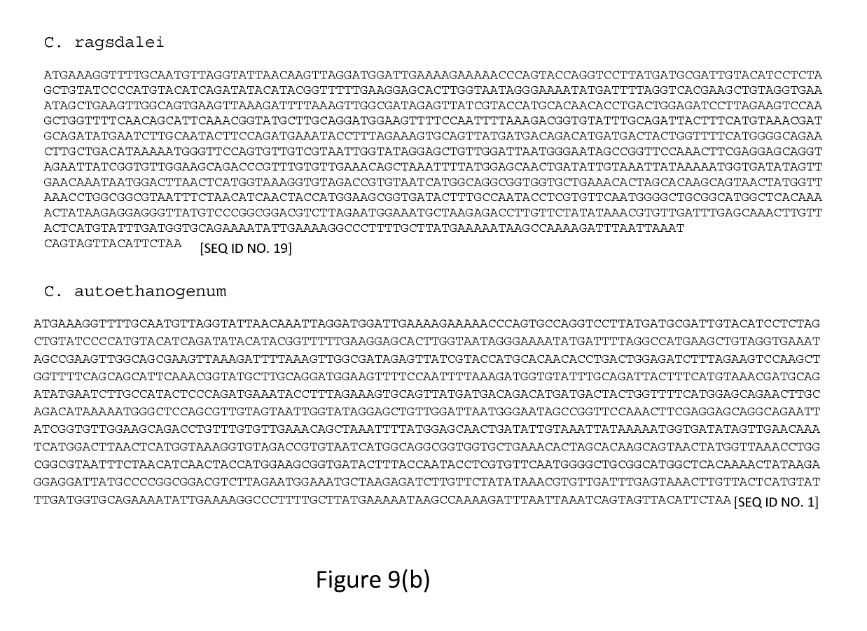

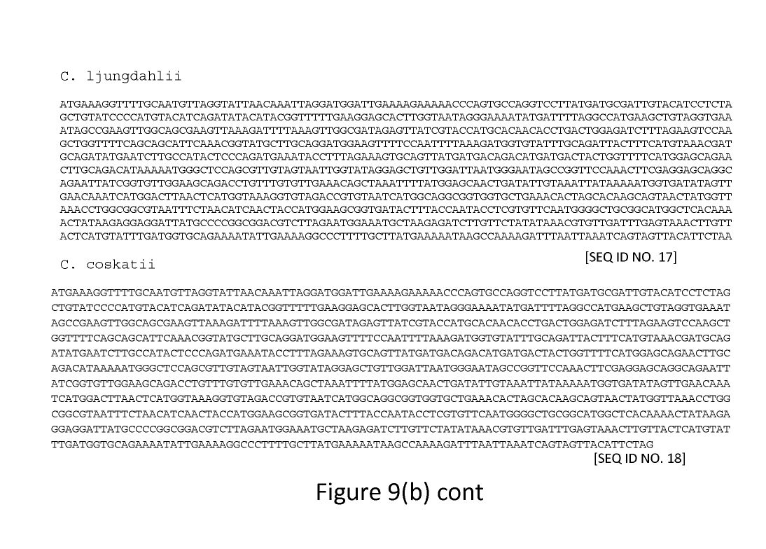

Examination of the genome sequences of Clostridia that exclusively produce C₂ alcohols and acids such as C. autoethanogenum, C. ragsdalei, C. coskatii, and C. ljungdahlii indicated the presence of a novel NADPH-dependent CoAR but not the butyrate kinase and butyryl-CoA transferase genes. The NADPH CoAR gene has been cloned and expressed in E. coli and has been shown to convert acetone, butanone and pentanone to their corresponding secondary alcohols indicating that it accommodates a variety of short-chained (C₃-C₅) ketones. This strain can also presumably convert the short-chain CoAs to their corresponding primary alcohols. These clostridia, when grown as a pure culture, produce ethanol and acetate but when in the presence of butyrate-producing organisms, are able to convert the acid in the CoA form to butanol in a 4-electron reduction. An alignment of the novel NADPH CoAR are shown for four syngas-utilizing homoacetogens (FIG. 9A). The raw sequences are shown in FIG. 9b. When the NADPH-dependent CoAR were aligned pairwise with Seq. ID No. 1 the percent identities were very high. C. ljungdahlii CoAR was 100% identical to Seq. ID No. 1 and 100% identical to CoAR of C. coskatii. The CoAR gene of C. ragsdalei showed 97.2% identity to Seq. ID No. 1.

### Example 8

### Example of Butanol Production in Single Stage Pilot Scale BCBR

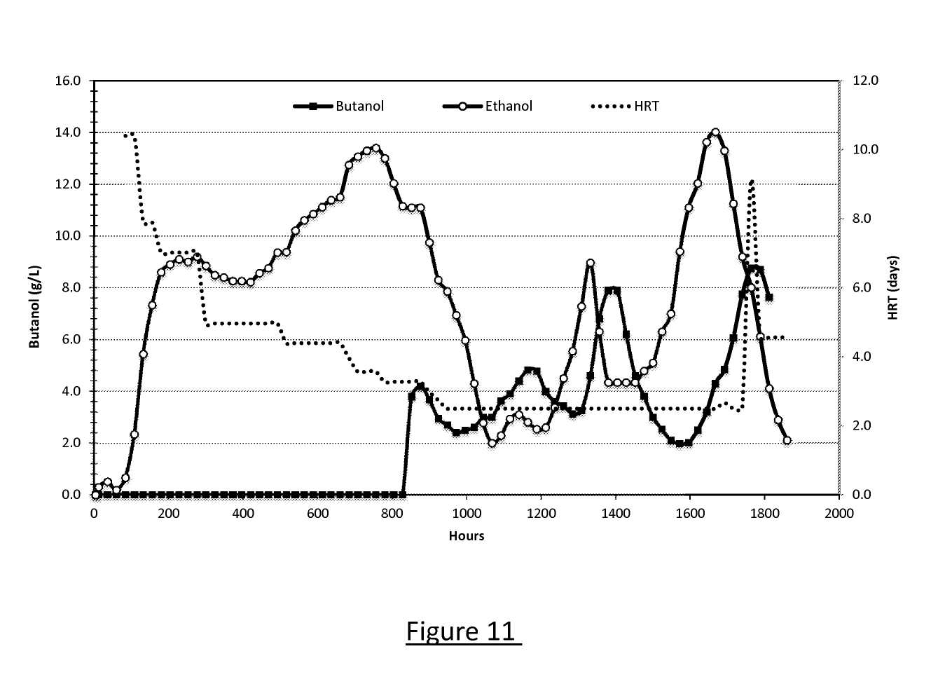

A 38,000 liter pilot scale Bubble Column BioReactor (BCBR) was first brought up to solventogenic conditions producing over 12 g/L of ethanol. The reactor was fed syngas as the only carbon and electron source to support the growth of the homoacetogen, Clostridium autoethanogenum. Composition of the syngas was on average, H₂-39, CO-29, CO₂-17, and CH₄-15 (mol %) and the rate of syngas addition varied from 35 to 144 lb/hr at a total fermenter volume of 26,000 liters. The HRT of the fermentation vessel was slowly stepped down from 8 days at the start of the fermentation to 3.3 days by 800 hours. FIG. 11 is a time plot of butanol and ethanol production and hydraulic retention time (HRT) from the 38,000 liter fermentor. After 800 hours, the ethanol producing fermentation was inoculated with a butyrogen culture.

The addition of the butyrogen culture and a further reduction of the HRT, showed an increase in the concentration of butanol (FIG. 11). Once initial butanol production was observed the HRT was further dropped to 2.5 days. Butanol concentrations rose and remained above 4 g/L and progressively rose as high as 8 g/L for the next 1000 hours under these conditions. Increasing the HRT to 9 days further increased the butanol concentration to 9 g/L total. The butanol concentration was the highest when the fermentation was at 2.5 days HRT Electron flow from syngas consumption to fermentation products during this 1000 hour period show that 60-80% of the electrons ended up as butanol product.

### Example 9

### Alignment and Percent Identity of a BCoATT Region Covered by One of the Detection Probes

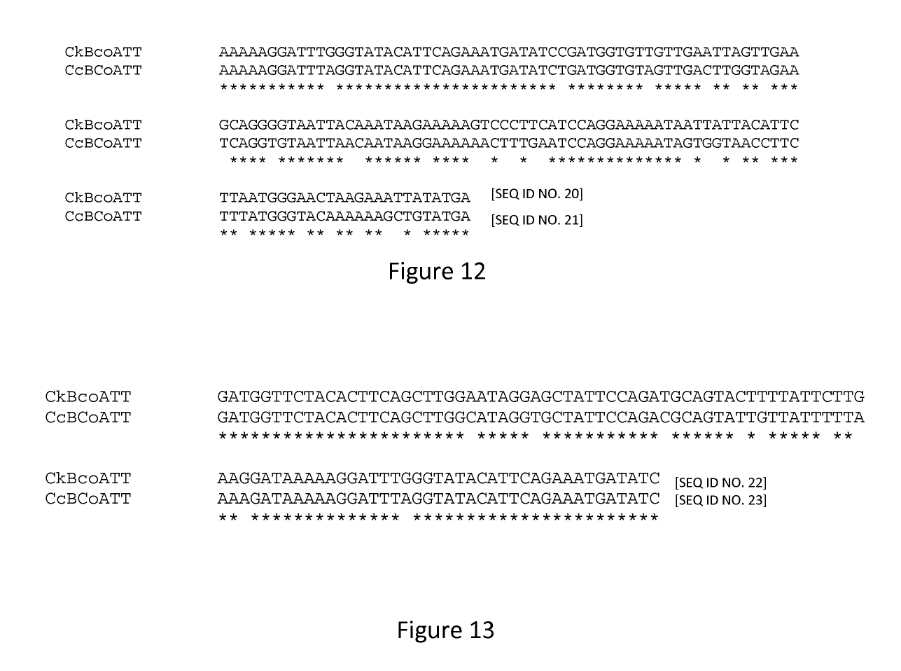

The PCR primers used in detecting butyrogens in different consortia (FIGS. 5a and 5b) covered a 145 bp region in the C. kluyveri and C. carboxidivorans butyryl-CoA acetate transferase gene (BCoATT). When the two Clostridial DNA sequences of this region were aligned the identity was determined to be 80% (FIG. 12). This indicates that the probe targeted a well conserved region that will be useful for detecting not only C. kluyveri type butyrogens that use ethanol and acetate to make butyrate but also heteroacetogens which can use syngas as substrate to make C₄ compounds. Several PCR reactions were run using pure C. kluyveri and pure C. carboxidivorans genomic DNA and both gave very strong amplicons of the expected size (data not shown).

### Example 10

### Alignment and Percent Identity of a BCoATT Region Covered by a Second Butyrogen Detection Probe

A second BCoATT detection probe was generated that covered a 101 bp region of BCoATT (Seq. ID No. 2) different from the one described in Example 9. Alignment of the two regions in the BCoATT genes showed the identity to be 91% (FIG. 13). This probe was also used to detect butyrogens in consortium samples and with pure genomic DNA isolated from C. kluyveri and C. carboxidivorans. All pure DNA and consortium samples gave a strong amplicon of the expected size (data not shown). This probe has been used along with the BCoATT probe described in Example 10 to provide a powerful detection tool for monitoring butyrogen populations in syntrophic butanol-producing reactors.

### Example 11

### Alignment and Percent Identity of a BuK Region Covered by One of the Detection Probes

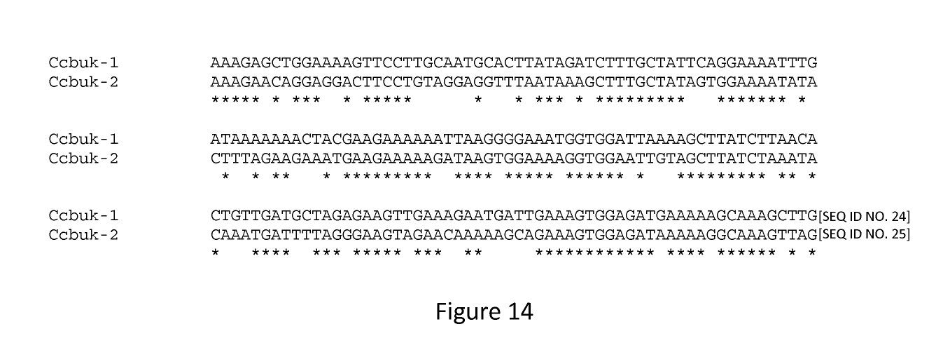

The PCR primers used in detecting butyrogens in different consortia in FIGS. 6a and 6b covered a 180 bp region in the C. carboxidivorans butyrate kinase #1 gene (Buk) (Seq. ID No. 3). When BuK#1 (Seq. ID #3) and the 180 bp region covered by the PCR probe (Seq. ID No. 8) were aligned the identity was determined to be 68% (FIG. 14). This indicates that the probe targets a well moderately conserved region that will be useful for detecting butyrate kinase-containing butyrogens. Several PCR reactions were run using pure C. carboxidivorans genomic DNA and both gave very strong amplicons of the expected size (data not shown).

### Example 12

### Alignment and Percent Identity of a BuK Gene Between Two Butyrogenic Organisms

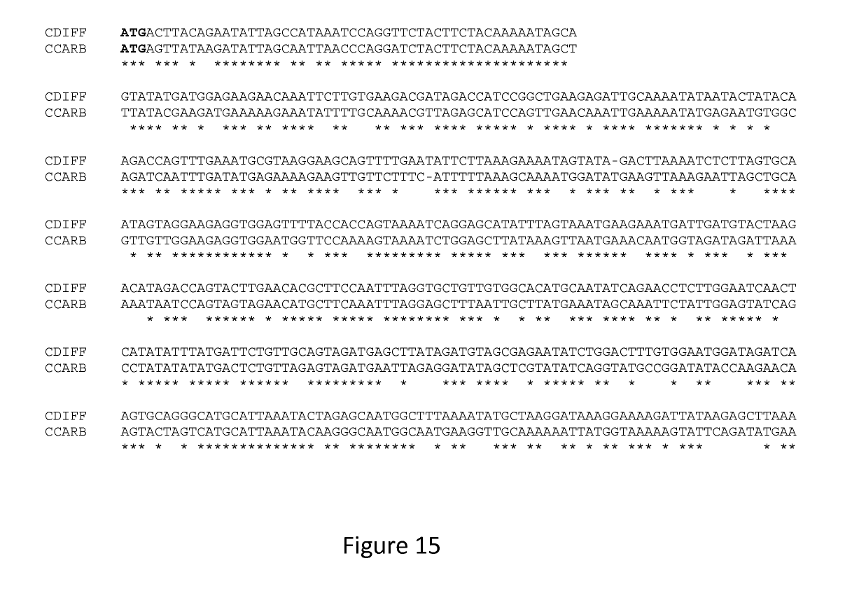

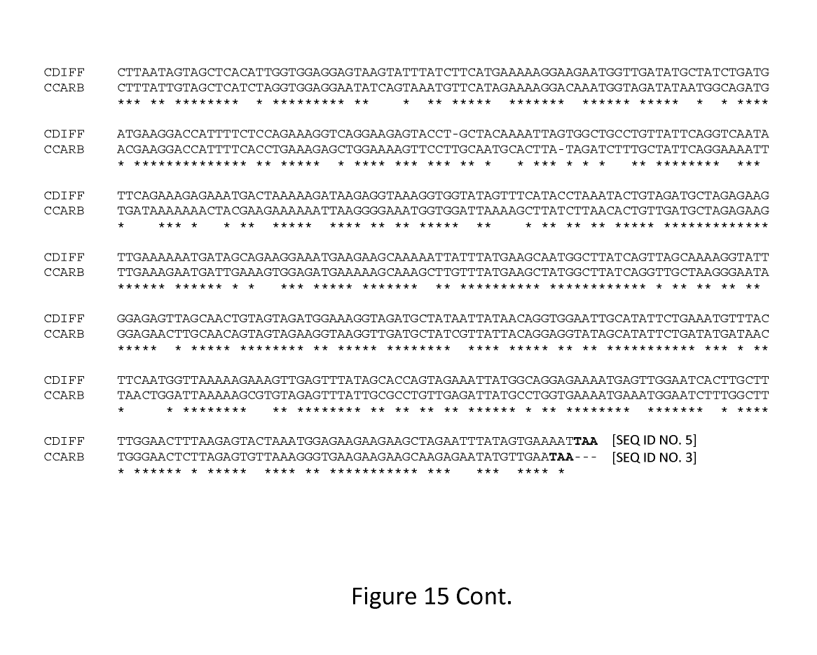

Alignment of butyrate kinase genes from C. carboxidivorans which is a syngas-fermenting butyrogen and C. difficile, which is primarily a carbohydrate-fermenting organism shows a 70.8% identity (FIG. 15). This shows that the butyrate kinase has a relatively high degree of conserved nucleotides across two highly unrelated butyrogenic organisms.

### Sequence Listings:

Seq. ID#1
C. autoethanogenum NADPH-dependent acetyl-CoA reductase
ATGAAAGGTTTTGCAATGTTAGGTATTAACAAATTAGGATGGATTGAAAAGAAAAACCCAGTGCCAGGTCCTTATGATGCGATTGTACATCCTCTA
GCTGTATCCCCATGTACATCAGATATACATACGGTTTTTGAAGGAGCACTTGGTAATAGGGAAAATATGATTTTAGGCCATGAAGCTGTAGGTGAA
ATAGCCGAAGTTGGCAGCGAAGTTAAAGATTTTAAAGTTGGCGATAGAGTTATCGTACCATGCACAACACCTGACTGGAGATCTTTAGAAGTCCAA
GCTGGTTTTCAGCAGCATTCAAACGGTATGCTTGCAGGATGGAAGTTTTCCAATTTTAAAGATGGTGTATTTGCAGATTACTTTCATGTAAACGAT
GCAGATATGAATCTTGCCATACTCCCAGATGAAATACCTTTAGAAAGTGCAGTTATGATGACAGACATGATGACTACTGGTTTTCATGGAGCAGAA
CTTGCAGACATAAAAATGGGCTCCAGCGTTGTAGTAATTGGTATAGGAGCTGTTGGATTAATGGGAATAGCCGGTTCCAAACTTCGAGGAGCAGGC
AGAATTATCGGTGTTGGAAGCAGACCTGTTTGTGTTGAAACAGCTAAATTTTATGGAGCAACTGATATTGTAAATTATAAAAATGGTGATATAGTT
GAACAAATCATGGACTTAACTCATGGTAAAGGTGTAGACCGTGTAATCATGGCAGGCGGTGGTGCTGAAACACTAGCACAAGCAGTAACTATGGTT
AAACCTGGCGGCGTAATTTCTAACATCAACTACCATGGAAGCGGTGATACTTTACCAATACCTCGTGTTCAATGGGGCTGCGGCATGGCTCACAAA
ACTATAAGAGGAGGATTATGCCCCGGCGGACGTCTTAGAATGGAAATGCTAAGAGATCTTGTTCTATATAAACGTGTTGATTTGAGTAAACTTGTT
ACTCATGTATTTGATGGTGCAGAAAATATTGAAAAGGCCCTTTTGCTTATGAAAAATAAGCCAAAAGATTTAATTAAATCAGTAGTTACATTCTAA
Seq. ID#2
C. kluyveri Butyryl-CoA acetate transferase #1
ATGGTTTTTAAAAATTGGCAGGATCTTTATAAAAGTAAAATTGTTAGTGCAGACGAAGCTGTATCTAAAGTAAGCTGTGGAGATAGCATAATTTTA
GGCAATGCTTGTGGAGCATCTCTTACACTTTTAGATGCCTTGGCTGCAAATAAGGAAAAGTATAAGAGTGTAAAGATACACAATCTTATACTTAAT
TATAAAAATGATATATATACTGATCCGGAATCAGAAAAGTATATTCATGGAAATACTTTCTTTGTAAGTGGAGGTACAAAGGAAGCAGTTAATTGT
AATAGAACAGATTATACTCCATGCTTTTTTTATGAAATACCAAAATTATTAAAACAAAAGTATATAAATGCAGATGTAGCTTTTATTCAAGTAAGT
AAGCCTGATAGCCATGGATACTGTAGCTTTGGAGTATCAACCGATTATTCACAGGCAATGGTACAGTCTGCAAAGCTTATAATTGCAGAAGTAAAC
GATCAGATGCCAAGAGTTTTAGGAGACAATTTTATACACATTTCTGATATGGATTACATAGTAGAAAGTTCACGTCCAATTCTAGAATTGACTCCT
CCTAAAATAGGAGAAGTAGAGAAGACAATAGGAAAATACTGTGCATCTCTTGTAGAAGATGGTTCTACACTTCAGCTTGGAATAGGAGCTATTCCA
GATGCAGTACTTTTATTCTTGAAGGATAAAAAGGATTTGGGTATACATTCAGAAATGATATCCGATGGTGTTGTTGAATTAGTTGAAGCAGGGGTA
ATTACAAATAAGAAAAAGTCCCTTCATCCAGGAAAAATAATTATTACATTCTTAATGGGAACTAAGAAATTATATGATTTCATAAATGATAATCCT
ATGGTAGAAGGATACCCTGTAGATTATGTAAATGATCCTAAGGTTATTATGCAAAATTCTAAGATGGTATGTATAAACTCCTGTGTAGAAGTGGAT
TTCACAGGACAAGTGTGTGCTGAAAGTGTAGGATTTAAACAAATAAGCGGTGTAGGTGGACAAGTTGATTACATGAGAGGAGCTAGCATGGCTGAT
GGAGGAAAATCAATTCTTGCTATACCATCTACTGCAGCTGGCGGCAAAATTTCAAGAATAGTTCCTATTTTAACTGAAGGAGCGGGGGTTACTACT
TCAAGATATGATGTTCAATATGTTGTTACAGAATATGGTATTGCACTTCTCAAGGGCAAATCCATAAGAGAAAGAGCTAAGGAGCTTATAAAAATT
GCACATCCTAAATTTAGGGAAGAATTAACAGCTCAATTTGAAAAAAGATTCAGTTGTAAGCTTTAA
Seq. ID#3
C. carboxidivorans Butyrate kinase #1
ATGAGTTATAAGATATTAGCAATTAACCCAGGATCTACTTCTACAAAAATAGCTTTATACGAAGATGAAAAAGAAATATTTTGCAAAACGTTAGAG
CATCCAGTTGAACAAATTGAAAAATATGAGAATGTGGCAGATCAATTTGATATGAGAAAAGAAGTTGTTCTTTCATTTTTAAAGCAAAATGGATAT
GAAGTTAAAGAATTAGCTGCAGTTGTTGGAAGAGGTGGAATGGTTCCAAAAGTAAAATCTGGAGCTTATAAAGTTAATGAAACAATGGTAGATAGA
TTAAAAAATAATCCAGTAGTAGAACATGCTTCAAATTTAGGAGCTTTAATTGCTTATGAAATAGCAAATTCTATTGGAGTATCAGCCTATATATAT
GACTCTGTTAGAGTAGATGAATTAGAGGATATAGCTCGTATATCAGGTATGCCGGATATACCAAGAACAAGTACTAGTCATGCATTAAATACAAGG
GCAATGGCAATGAAGGTTGCAAAAAATTATGGTAAAAAGTATTCAGATATGAACTTTATTGTAGCTCATCTAGGTGGAGGAATATCAGTAAATGTT
CATAGAAAAGGACAAATGGTAGATATAATGGCAGATGACGAAGGACCATTTTCACCTGAAAGAGCTGGAAAAGTTCCTTGCAATGCACTTATAGAT
CTTTGCTATTCAGGAAAATTTGATAAAAAAACTACGAAGAAAAAATTAAGGGGAAATGGTGGATTAAAAGCTTATCTTAACACTGTTGATGCTAGA
GAAGTTGAAAGAATGATTGAAAGTGGAGATGAAAAAGCAAAGCTTGTTTATGAAGCTATGGCTTATCAGGTTGCTAAGGGAATAGGAGAACTTGCA
ACAGTAGTAGAAGGTAAGGTTGATGCTATCGTTATTACAGGAGGTATAGCATATTCTGATATGATAACTAACTGGATTAAAAAGCGTGTAGAGTTT
ATTGCGCCTGTTGAGATTATGCCTGGTGAAAATGAAATGGAATCTTTGGCTTTGGGAACTCTTAGAGTGTTAAAGGGTGAAGAAGAAGCAAGAGAA
TATGTTGAATAA
Seq.ID #4
C. carboxidivorans butyrate kinase #2
TTGCTAATTAAAATATTTATTAAGTATTGCTATAATCAGGAGGGTAAAATAATGTACAAAATACTAGCAATAAATCCAGGTTCAACTTCAACTAAA
ATAGCTATTTATGATGACACAGAGGAATTATTTAAAACCACTATAGAACATTCTAGTGAAGAAGTGAAAAAATATGAAAACATAGCTGATCAATAT
AGTATGAGATATGAAGCTATAATGAAATTTTTAAAAGAAGTAGATTTTGATGTCAAAGCTTTATCTGCAGTAGTTGGAAGAGGAGGAATTCTGCCT
CCAGTTAAATCAGGAGCTTACAGAGTAAATGATTCTATGGTAGAAAGACTGGCTAAAAGACCTGTAGTAGAGCATGCTTCAAATTTAGGAGCTATA
ATTTCATATGCAATAGCAAAACCTTTAAATATACCAGCTTTCATATATGATTCTGTAGCTGTAGATGAATTTGAGGATATTGCAAGAATATCAGGA
CTTGCAGATATAAAAAGAGAGAGTTTTATTCATGCTTTAAATATGAGAGCTGCAGCAATAAAAACAGCAAAAAAACTAGGTAAACCTTATGAACAA
TGTAATTTAGTTGTTGCTCATTTAGGAGGCGGAATATCTCTTACTGTACATAAAGGTGGAAAAATGATAGACGCTGTTACTGATGAAGAAGGACCG
TTTTCACCAGAAAGGTCAGGTAGAGTACCTTGTAAGCGCTTAATAGAAATGTGTTATAAAAATGATGAACGCACAATGAAAAAGAAAATAAGAGGA
GATGGTGGATTAATCTCTTATTTAGGAACTAATAGTGCATTAGATGTAGAAAAAAGAATTGAAAATGGAGATGCTGAAGCCAAATTAGTTTATGAA
GCTATGGCATATCAAATTGCAAAAGCAATAGGAGAACTTGCAACTGTAGTAAAGGGAAAGGTTGATGCAGTAGTAATTACAGGGGGAATTGCCTAT
TCAAAAATGATGACAGGATGGATAAAAGAAAGAGTAGAATTTATAGCACCTGTAGAGATATTGCCAGGAGAAAATGAATTAGAATCTCTTGCTTTA
GGTACGCTTAGAGTTATAAAGGGAGAAGAAAAAGCACACGAATATGATTTAGATTAG
Seq. ID #5
C. difficile butyrate kinase
ATGACTTACAGAATATTAGCCATAAATCCAGGTTCTACTT
CTACAAAAATAGCAGTATATGATGGAGAAGAACAAATTCTTGTGAAGACGATAGACCATCCGGCTGAAGAGATTGCAAAATATAATACTATACAAG
ACCAGTTTGAAATGCGTAAGGAAGCAGTTTTGAATATTCTTAAAGAAAATAGTATAGACTTAAAATCTCTTAGTGCAATAGTAGGAAGAGGTGGAG
TTTTACCACCAGTAAAATCAGGAGCATATTTAGTAAATGAAGAAATGATTGATGTACTAAGACATAGACCAGTACTTGAACACGCTTCCAATTTAG
GTGCTGTTGTGGCACATGCAATATCAGAACCTCTTGGAATCAACTCATATATTTATGATTCTGTTGCAGTAGATGAGCTTATAGATGTAGCGAGAA
TATCTGGACTTTGTGGAATGGATAGATCAAGTGCAGGGCATGCATTAAATACTAGAGCAATGGCTTTAAAATATGCTAAGGATAAAGGAAAAGATT
ATAAGAGCTTAAACTTAATAGTAGCTCACATTGGTGGAGGAGTAAGTATTTATCTTCATGAAAAAGGAAGAATGGTTGATATGCTATCTGATGATG
AAGGACCATTTTCTCCAGAAAGGTCAGGAAGAGTACCTGCTACAAAATTAGTGGCTGCCTGTTATTCAGGTCAATATTCAGAAAGAGAAATGACTA
AAAAGATAAGAGGTAAAGGTGGTATAGTTTCATACCTAAATACTGTAGATGCTAGAGAAGTTGAAAAAATGATAGCAGAAGGAAATGAAGAAGCAA
AAATTATTTATGAAGCAATGGCTTATCAGTTAGCAAAAGGTATTGGAGAGTTAGCAACTGTAGTAGATGGAAAGGTAGATGCTATAATTATAACAG
GTGGAATTGCATATTCTGAAATGTTTACTTCAATGGTTAAAAAGAAAGTTGAGTTTATAGCACCAGTAGAAATTATGGCAGGAGAAAATGAGTTGG
ATAATCACTTGCTTTTGGAACTTTAAGAGTACTAAATGGAGAAGAAGAAGCTAGAATTTATAGTGAAA
Seq. ID#6
145 bp region covered by BCoATT probe contained in Seq. ID No. 2
AAAAAGGATTTGGGTATACATTCAGAAATGATATCCGATGGTGTTGTTGAATTAGTTGAAGCAGGGGTAATTACAAATAAGAAAAAGTCCCTTCATC
CAGGAAAAATAATTATTACATTCTTAATGGGAACTAAGAAATTATATGA
Seq. ID #7
101 bp region covered by BCoATT probe contained in Seq. ID No. 2
GATGGTTCTACACTTCAGCTTGGAATAGGAGCTATTCCAGATGCAGTACTTTTATTCTTGAAGGATAAAAAGGATTTGGGTATACATTCAGAAATGA
TATC
Seq. ID #8
180 bp region covered by Buk probe contained in Seq. ID No. 3
AAAGAGCTGGAAAAGTTCCTTGCAATGCACTTATAGATCTTTGCTATTCAGGAAAATTTGATAAAAAAACTACGAAGAAAAAATTAAGGGGAAATGG
TGGATTAAAAGCTTATCTTAACACTGTTGATGCTAGAGAAGTTGAAAGAATGATTGAAAGTGGAGATGAAAAAGCAAAGCTTG
Seq. ID #9
Butyrate Kinase identified in C. carboxidivorans.
ATGTCATATAAATTATTAATATTAAATCCAGGATCTACATCTACCAAAATAGGAGTATATGATGGAGAAAATGAAATTTTAGAAGAAACTTTAAGA
CATTCTTCAGAAGAAATTGAGAAATATGCTACTATTTATGATCAATTTGAATTTAGAAAAGAAGTTATATTGAAGGTTTTAAAAGAAAAGAATTTT
GATATTAATACATTAGACGGAGTAGTAGGCAGAGGTGGATTATTAAAACCAATTGAAAGTGGAACTTATAAAGTCAATGATGCTATGTTAGAAGAC
CTAAAAGTTGGAGTGCAAGGACAGCATGCTTCAAATTTAGGTGGAATAATAGCTAATGAAATAGGAAAATCTATAAATAAACCAGCATTTATAGTA
GACCCAGTTGTTGTTGATGAATTAGATGAAGCAGCTAGAATATCCGGAATGCCTGAAATAGAAAGAATAAGTATATTCCATGCTTTAAATCAAAAA
GCAGTAGCAAAGAGATATGCAAAAGAAAACAATAAGAAGTATGATGAATTAAATTTAGTAGTGACACACATGGGTGGCGGAGTAACTGTTGGAGCT
CACAAAAAAGGAAGAGTTGTAGATGTAGCCAATGGTTTAGATGGAGATGGACCATTTTCACCAGAAAGAACAGGAGGACTTCCTGTAGGAGGTTTA
ATAAAGCTTTGCTATAGTGGAAAATATACTTTAGAAGAAATGAAGAAAAAGATAAGTGGAAAAGGTGGAATTGTAGCTTATCTAAATACAAATGAT
TTTAGGGAAGTAGAACAAAAAGCAGAAAGTGGAGATAAAAAGGCAAAGTTAGTATTTGATGCTTTCATATTACAAGTAGGTAAAGAAATTGGTAAA
TGTGCTGCAGTTTTACATGGAAAAGTAGATGCTTTAATTTTAACTGGAGGAATAGCTTATAGTAAAACTGTTACAGCTGCAATAAAAGACATGGTA
GAATTTATTGCACCAGTTGTAGTTTATCCAGGAGAAGATGAATTATTAGCATTAGCACAAGGCGGACTTAGAGTACTAGGTGGAGAAGAACAAGCA
AAAGAATATAAGTAA

1. A microorganism co-culture for the conversion of at least one of CO or CO₂ and H₂ to butanol said co-culture comprising two or more microorganisms collectively having a nucleotide sequence identity at least 95% identical to SEQ ID No. 1 and a nucleotide sequence identity at least 70% identical to SEQ ID No. 2 or at least 65% identical to SEQ. ID No. 3.

2. The co-culture of claim 1 wherein the co-culture has a nucleotide sequence identity at least 75% identical to SEQ ID No. 2.

3. The co-culture of claim 1 wherein the co-culture has a nucleotide sequence identity at least 75% identical to SEQ ID No. 2, and a nucleotide sequence identity at least 71% identical to SEQ. ID No. 3.

4. The co-culture of claim 1 wherein the co-culture includes C. kluyveri.

5. The co-culture of claim 1 wherein the co-culture includes one or more homoacetogenic microorganisms selected from the group consisting of C. ljungdahlii, C. ragsdaeli, C. authoethanongenum and C. coskatii

6. The co-culture of claim 5 wherein the co-culture comprises a mixture of a homoacetogenic microorganism and a butyrogenic microorganism.

7. The co-culture of claim 6 wherein the homocetogenic microorganism is cultured in a fermentor until it produces a concentration of ethanol of at least 1 g/L and the butyrogenic microorganism is added to the fermentor to produce the microorganism co-culture.

8. A microorganism co-culture having a nucleotide sequence defining a gene for NADPH dependent CoA reductase and a nucleotide sequence defining a gene for at least one of a Butyryl-CoA acetate transferase and Butyrate kinase.

9. The co-culture of claim 8 wherein the co-culture has a nucleotide sequence defining a gene for Butyryl-CoA acetate transferase.

10. The co-culture of claim 9 wherein the co-culture has a nucleotide sequence defining a gene for Butyryl-CoA acetate transferase and defining a gene for Butyrate kinase.

11. The co-culture of claim 8 wherein the co-culture includes C. kluyveri.

12. The co-culture of claim 8 wherein the co-culture includes one or more homoacetogenic microorganisms selected from the group consisting of C. ljungdahlii, C. ragsdaeli, C. authoethanongenum and C. coskatii

13. The co-culture of claim 12 wherein the co-culture comprises a mixture of a homoacetogenic microorganism and a butyrogenic microorganism.

14. The co-culture of claim 13 wherein the homocetogenic microorganism is cultured in a fermentor until it produces a concentration of ethanol of at least 10 g/L and the butyrogenic microorganism is added to the fermentor to produce the microorganism co-culture.


SEQUENCE LISTING (31 sequences)

SEQ ID NO: 1 - DNA, 1056 bp, Clostridium autoethanogenum
atgaaaggttttgcaatgttaggtattaacaaattaggatggattgaaaagaaaaacccagtgccaggtccttatgatgcgattgtacatcctctagctgtatccccatgtacatcagatatacatacggtttttgaaggagcacttggtaatagggaaaatatgattttaggccatgaagctgtaggtgaaatagccgaagttggcagcgaagttaaagattttaaagttggcgatagagttatcgtaccatgcacaacacctgactggagatctttagaagtccaagctggttttcagcagcattcaaacggtatgcttgcaggatggaagttttccaattttaaagatggtgtatttgcagattactttcatgtaaacgatgcagatatgaatcttgccatactcccagatgaaatacctttagaaagtgcagttatgatgacagacatgatgactactggttttcatggagcagaacttgcagacataaaaatgggctccagcgttgtagtaattggtataggagctgttggattaatgggaatagccggttccaaacttcgaggagcaggcagaattatcggtgttggaagcagacctgtttgtgttgaaacagctaaattttatggagcaactgatattgtaaattataaaaatggtgatatagttgaacaaatcatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttaccaatacctcgtgttcaatggggctgcggcatggctcacaaaactataagaggaggattatgccccggcggacgtcttagaatggaaatgctaagagatcttgttctatataaacgtgttgatttgagtaaacttgttactcatgtatttgatggtgcagaaaatattgaaaaggcccttttgcttatgaaaaataagccaaaagatttaattaaatcagtagttacattctaa

SEQ ID NO: 2 - DNA, 1314 bp, Clostridium kluyveri
atggtttttaaaaattggcaggatctttataaaagtaaaattgttagtgcagacgaagctgtatctaaagtaagctgtggagatagcataattttaggcaatgcttgtggagcatctcttacacttttagatgccttggctgcaaataaggaaaagtataagagtgtaaagatacacaatcttatacttaattataaaaatgatatatatactgatccggaatcagaaaagtatattcatggaaatactttctttgtaagtggaggtacaaaggaagcagttaattgtaatagaacagattatactccatgctttttttatgaaataccaaaattattaaaacaaaagtatataaatgcagatgtagcttttattcaagtaagtaagcctgatagccatggatactgtagctttggagtatcaaccgattattcacaggcaatggtacagtctgcaaagcttataattgcagaagtaaacgatcagatgccaagagttttaggagacaattttatacacatttctgatatggattacatagtagaaagttcacgtccaattctagaattgactcctcctaaaataggagaagtagagaagacaataggaaaatactgtgcatctcttgtagaagatggttctacacttcagcttggaataggagctattccagatgcagtacttttattcttgaaggataaaaaggatttgggtatacattcagaaatgatatccgatggtgttgttgaattagttgaagcaggggtaattacaaataagaaaaagtcccttcatccaggaaaaataattattacattcttaatgggaactaagaaattatatgatttcataaatgataatcctatggtagaaggataccctgtagattatgtaaatgatcctaaggttattatgcaaaattctaagatggtatgtataaactcctgtgtagaagtggatttcacaggacaagtgtgtgctgaaagtgtaggatttaaacaaataagcggtgtaggtggacaagttgattacatgagaggagctagcatggctgatggaggaaaatcaattcttgctataccatctactgcagctggcggcaaaatttcaagaatagttcctattttaactgaaggagcgggggttactacttcaagatatgatgttcaatatgttgttacagaatatggtattgcacttctcaagggcaaatccataagagaaagagctaaggagcttataaaaattgcacatcctaaatttagggaagaattaacagctcaatttgaaaaaagattcagttgtaagctttaa

SEQ ID NO: 3 - DNA, 1068 bp, Clostridium carboxidivorans
atgagttataagatattagcaattaacccaggatctacttctacaaaaatagctttatacgaagatgaaaaagaaatattttgcaaaacgttagagcatccagttgaacaaattgaaaaatatgagaatgtggcagatcaatttgatatgagaaaagaagttgttctttcatttttaaagcaaaatggatatgaagttaaagaattagctgcagttgttggaagaggtggaatggttccaaaagtaaaatctggagcttataaagttaatgaaacaatggtagatagattaaaaaataatccagtagtagaacatgcttcaaatttaggagctttaattgcttatgaaatagcaaattctattggagtatcagcctatatatatgactctgttagagtagatgaattagaggatatagctcgtatatcaggtatgccggatataccaagaacaagtactagtcatgcattaaatacaagggcaatggcaatgaaggttgcaaaaaattatggtaaaaagtattcagatatgaactttattgtagctcatctaggtggaggaatatcagtaaatgttcatagaaaaggacaaatggtagatataatggcagatgacgaaggaccattttcacctgaaagagctggaaaagttccttgcaatgcacttatagatctttgctattcaggaaaatttgataaaaaaactacgaagaaaaaattaaggggaaatggtggattaaaagcttatcttaacactgttgatgctagagaagttgaaagaatgattgaaagtggagatgaaaaagcaaagcttgtttatgaagctatggcttatcaggttgctaagggaataggagaacttgcaacagtagtagaaggtaaggttgatgctatcgttattacaggaggtatagcatattctgatatgataactaactggattaaaaagcgtgtagagtttattgcgcctgttgagattatgcctggtgaaaatgaaatggaatctttggctttgggaactcttagagtgttaaagggtgaagaagaagcaagagaatatgttgaataa

SEQ ID NO: 4 - DNA, 1113 bp, Clostridium carboxidivorans
ttgctaattaaaatatttattaagtattgctataatcaggagggtaaaataatgtacaaaatactagcaataaatccaggttcaacttcaactaaaatagctatttatgatgacacagaggaattatttaaaaccactatagaacattctagtgaagaagtgaaaaaatatgaaaacatagctgatcaatatagtatgagatatgaagctataatgaaatttttaaaagaagtagattttgatgtcaaagctttatctgcagtagttggaagaggaggaattctgcctccagttaaatcaggagcttacagagtaaatgattctatggtagaaagactggctaaaagacctgtagtagagcatgcttcaaatttaggagctataatttcatatgcaatagcaaaacctttaaatataccagctttcatatatgattctgtagctgtagatgaatttgaggatattgcaagaatatcaggacttgcagatataaaaagagagagttttattcatgctttaaatatgagagctgcagcaataaaaacagcaaaaaaactaggtaaaccttatgaacaatgtaatttagttgttgctcatttaggaggcggaatatctcttactgtacataaaggtggaaaaatgatagacgctgttactgatgaagaaggaccgttttcaccagaaaggtcaggtagagtaccttgtaagcgcttaatagaaatgtgttataaaaatgatgaacgcacaatgaaaaagaaaataagaggagatggtggattaatctcttatttaggaactaatagtgcattagatgtagaaaaaagaattgaaaatggagatgctgaagccaaattagtttatgaagctatggcatatcaaattgcaaaagcaataggagaacttgcaactgtagtaaagggaaaggttgatgcagtagtaattacagggggaattgcctattcaaaaatgatgacaggatggataaaagaaagagtagaatttatagcacctgtagagatattgccaggagaaaatgaattagaatctcttgctttaggtacgcttagagttataaagggagaagaaaaagcacacgaatatgatttagattag

SEQ ID NO: 5 - DNA, 1068 bp, Clostridium difficile
atgacttacagaatattagccataaatccaggttctacttctacaaaaatagcagtatatgatggagaagaacaaattcttgtgaagacgatagaccatccggctgaagagattgcaaaatataatactatacaagaccagtttgaaatgcgtaaggaagcagttttgaatattcttaaagaaaatagtatagacttaaaatctcttagtgcaatagtaggaagaggtggagttttaccaccagtaaaatcaggagcatatttagtaaatgaagaaatgattgatgtactaagacatagaccagtacttgaacacgcttccaatttaggtgctgttgtggcacatgcaatatcagaacctcttggaatcaactcatatatttatgattctgttgcagtagatgagcttatagatgtagcgagaatatctggactttgtggaatggatagatcaagtgcagggcatgcattaaatactagagcaatggctttaaaatatgctaaggataaaggaaaagattataagagcttaaacttaatagtagctcacattggtggaggagtaagtatttatcttcatgaaaaaggaagaatggttgatatgctatctgatgatgaaggaccattttctccagaaaggtcaggaagagtacctgctacaaaattagtggctgcctgttattcaggtcaatattcagaaagagaaatgactaaaaagataagaggtaaaggtggtatagtttcatacctaaatactgtagatgctagagaagttgaaaaaatgatagcagaaggaaatgaagaagcaaaaattatttatgaagcaatggcttatcagttagcaaaaggtattggagagttagcaactgtagtagatggaaaggtagatgctataattataacaggtggaattgcatattctgaaatgtttacttcaatggttaaaaagaaagttgagtttatagcaccagtagaaattatggcaggagaaaatgagttggataatcacttgcttttggaactttaagagtactaaatggagaagaagaagctagaatttatagtgaaa

SEQ ID NO: 6 - DNA, 146 bp, Clostridium kluyveri
aaaaaggatttgggtatacattcagaaatgatatccgatggtgttgttgaattagttgaagcaggggtaattacaaataagaaaaagtcccttcatccaggaaaaataattattacattcttaatgggaactaagaaattatatga

SEQ ID NO: 7 - DNA, 101 bp, Clostridium kluyveri
gatggttctacacttcagcttggaataggagctattccagatgcagtacttttattcttgaaggataaaaaggatttgggtatacattcagaaatgatatc

SEQ ID NO: 8 - DNA, 180 bp, Clostridium carboxidivorans
aaagagctggaaaagttccttgcaatgcacttatagatctttgctattcaggaaaatttgataaaaaaactacgaagaaaaaattaaggggaaatggtggattaaaagcttatcttaacactgttgatgctagagaagttgaaagaatgattgaaagtggagatgaaaaagcaaagcttg

SEQ ID NO: 9 - DNA, 1071 bp, Clostridium carboxidivorans
atgtcatataaattattaatattaaatccaggatctacatctaccaaaataggagtatatgatggagaaaatgaaattttagaagaaactttaagacattcttcagaagaaattgagaaatatgctactatttatgatcaatttgaatttagaaaagaagttatattgaaggttttaaaagaaaagaattttgatattaatacattagacggagtagtaggcagaggtggattattaaaaccaattgaaagtggaacttataaagtcaatgatgctatgttagaagacctaaaagttggagtgcaaggacagcatgcttcaaatttaggtggaataatagctaatgaaataggaaaatctataaataaaccagcatttatagtagacccagttgttgttgatgaattagatgaagcagctagaatatccggaatgcctgaaatagaaagaataagtatattccatgctttaaatcaaaaagcagtagcaaagagatatgcaaaagaaaacaataagaagtatgatgaattaaatttagtagtgacacacatgggtggcggagtaactgttggagctcacaaaaaaggaagagttgtagatgtagccaatggtttagatggagatggaccattttcaccagaaagaacaggaggacttcctgtaggaggtttaataaagctttgctatagtggaaaatatactttagaagaaatgaagaaaaagataagtggaaaaggtggaattgtagcttatctaaatacaaatgattttagggaagtagaacaaaaagcagaaagtggagataaaaaggcaaagttagtatttgatgctttcatattacaagtaggtaaagaaattggtaaatgtgctgcagttttacatggaaaagtagatgctttaattttaactggaggaatagcttatagtaaaactgttacagctgcaataaaagacatggtagaatttattgcaccagttgtagtttatccaggagaagatgaattattagcattagcacaaggcggacttagagtactaggtggagaagaacaagcaaaagaatataagtaa

SEQ ID NO: 10 - DNA, 1334 bp, Clostridium kluyveri
tttgaaaggtggtttttagaatggtttttaaaaattggcaggatctttataaaagtaaaattgttagtgcagacgaagctgtatctaaagtaagctgtggagatagcataattttaggcaatgcttgtggagcatctcttacacttttagatgccttggctgcaaataaggaaaagtataagagtgtaaagatacacaatcttatacttaattataaaaatgatatatatactgatccggaatcagaaaagtatattcatggaaatactttctttgtaagtggaggtacaaaggaagcagttaattgtaatagaacagattatactccatgctttttttatgaaataccaaaattattaaaacaaaagtatataaatgcagatgtagcttttattcaagtaagtaagcctgatagccatggatactgtagctttggagtatcaaccgattattcacaggcaatggtacagtctgcaaagcttataattgcagaagtaaacgatcagatgccaagagttttaggagacaattttatacacatttctgatatggattacatagtagaaagttcacgtccaattctagaattgactcctcctaaaataggagaagtagagaagacaataggaaaatactgtgcatctcttgtagaagatggttctacacttcagcttggaataggagctattccagatgcagtacttttattcttgaaggataaaaaggatttgggtatacattcagaaatgatatccgatggtgttgttgaattagttgaagcaggggtaattacaaataagaaaaagtcccttcatccaggaaaaataattattacattcttaatgggaactaagaaattatatgatttcataaatgataatcctatggtagaaggataccctgtagattatgtaaatgatcctaaggttattatgcaaaattctaagatggtatgtataaactcctgtgtagaagtggatttcacaggacaagtgtgtgctgaaagtgtaggatttaaacaaataagcggtgtaggtggacaagttgattacatgagaggagctagcatggctgatggaggaaaatcaattcttgctataccatctactgcagctggcggcaaaatttcaagaatagttcctattttaactgaaggagcgggggttactacttcaagatatgatgttcaatatgttgttacagaatatggtattgcacttctcaagggcaaatccataagagaaagagctaaggagcttataaaaattgcacatcctaaatttagggaagaattaacagctcaatttgaaaaaagattcagttgtaagctttaa

SEQ ID NO: 11 - DNA, 1335 bp, Clostridium carboxidivorans
taaacgtattagtgtatagaaaggatgatctttatggattggaaaaaactatataaaagtaaattagtaagtgcaaaagaagcagtttctaatataaagtctaatagtagagttgttgtaagcattgcagttgcagaacctacagagcttatagatgcgttggtagcaaataaagaaaattatgaaaatgtggaagtagtgcatatggtagatatgggaaagagcgaatatgcacaagaaggaatggaaaagtattttaaatataattccatatttgtgggtgctagtaccaaggatgctgttaattctggaagatcagattttactccatgttgtttttatgaattacctagattatttgaagagggatatttacctgtagatgtggtattaattcaagtaagtaagcctgataaacatggctattgtagttttggagtatctaatgattatactaagccagcagcagactgtgctaaaatggtaattgcagaggtaaatgaaaatatgcctagagttttaggagattcatttatacatatatcagatattgattatatagtagaaacttcacatcctataatggagttaaaacaacctaagataggaaagattgaagaagctataggggaatattgtgcttcattgattgaggatggttctacacttcagcttggcataggtgctattccagacgcagtattgttatttttaaaagataaaaaggatttaggtatacattcagaaatgatatctgatggtgtagttgacttggtagaatcaggtgtaattaacaataaggaaaaaactttgaatccaggaaaaatagtggtaaccttctttatgggtacaaaaaagctgtatgattttatagatgataatcctatggtagagtcttaccctgtaagttatgtaaatgatcctacagttattatgaaaaactctaaaatgatttcaataaattcctgtgttgaggtggatttaatgggacaagtatgttctgaaagcataggaatgaaccagataagtggaattggaggacaggttgattttataagaggagctaatatgtgcaaggatggaaaagctattattgctataccatccactgctgctaaaggaaaagtttctagaatagttcctttaatagaaaaaggtacacctattacaacttccagaacagatgtagattacattattacagaatatggtattgctagacttaagagtaaatctttaaaagaaagagctagggctttgataaacattgctcatccagactttagagcatggcttatagatgaatatgaaaaaaggtttaaaactaaattttaa

SEQ ID NO: 12 - DNA, 1311 bp, Clostridium carboxidivorans
atgatctttatggattggaaaaaactatataaaagtaaattagtaagtgcaaaagaagcagtttctaatataaagtctaatagtagagttgttgtaagcattgcagttgcagaacctacagagcttatagatgcgttggtagcaaataaagaaaattatgaaaatgtggaagtagtgcatatggtagatatgggaaagagcgaatatgcacaagaaggaatggaaaagtattttaaatataattccatatttgtgggtgctagtaccaaggatgctgttaattctggaagatcagattttactccatgttgtttttatgaattacctagattatttgaagagggatatttacctgtagatgtggtattaattcaagtaagtaagcctgataaacatggctattgtagttttggagtatctaatgattatactaagccagcagcagactgtgctaaaatggtaattgcagaggtaaatgaaaatatgcctagagttttaggagattcatttatacatatatcagatattgattatatagtagaaacttcacatcctataatggagttaaaacaacctaagataggaaagattgaagaagctataggggaatattgtgcttcattgattgaggatggttctacacttcagcttggcataggtgctattccagacgcagtattgttatttttaaaagataaaaaggatttaggtatacattcagaaatgatatctgatggtgtagttgacttggtagaatcaggtgtaattaacaataaggaaaaaactttgaatccaggaaaaatagtggtaaccttctttatgggtacaaaaaagctgtatgattttatagatgataatcctatggtagagtcttaccctgtaagttatgtaaatgatcctacagttattatgaaaaactctaaaatgatttcaataaattcctgtgttgaggtggatttaatgggacaagtatgttctgaaagcataggaatgaaccagataagtggaattggaggacaggttgattttataagaggagctaatatgtgcaaggatggaaaagctattattgctataccatccactgctgctaaaggaaaagtttctagaatagttcctttaatagaaaaaggtacacctattacaacttccagaacagatgtagattacattattacagaatatggtattgctagacttaagagtaaatctttaaaagaaagagctagggctttgataaacattgctcatccagactttagagcatggcttatagatgaatatgaaaaaaggtttaaaactaaattttaa

SEQ ID NO: 13 - DNA, 1290 bp, Clostridium kluyveri
atggagtgggaagagatatataaagagaaactggtaactgcagaaaaagctgtttcaaaaatagaaaaccatagcagggtagtttttgcacatgcagtaggagaacccgtagatttagtaaatgcactagttaaaaataaggataattatataggactagaaatagttcacatggtagctatgggcaaaggtgaatatacaaaagagggtatgcaaagacattttagacataatgctttatttgtaggcggatgtactagagatgcagtaaattcaggaagagcagattatacaccttgttttttctatgaagtgccaagtttgtttaaagaaaaacgtttgcctgtagatgtagcacttattcaggtaagtgagccagataaatatggctactgcagttttggagtttccaatgactataccaagccagcagcagaaagtgctaagcttgtaattgcagaagtgaataaaaacatgccaagaactcttggagattcttttatacatgtatcagatattgattatatagtggaagcttcacacccattgttagaattgcagcctcctaaattgggagatgtagaaaaagccataggagaaaactgtgcatctttaattgaagatggagctactcttcagcttggaataggtgctataccagatgcggtacttttattcttaaagaacaaaaagaatttaggaatacattctgagatgatatcagatggtgtgatggaactggtgaaggcaggggttatcaataacaagaaaaagaccctccatccaggcaaaatagttgtaacatttttaatgggaacaaaaaaattatatgattttgtaaacaataatccaatggtagaaacttattctgtagattatgtaaataatccactggtaattatgaaaaatgacaatatggtttcaataaattcttgtgttcaagtagacttaatgggacaagtatgttctgaaagtataggattgaaacagataagtggagtgggaggccaggtagattttattagaggagctaatctatcaaagggtggaaaggctattatagctataccttccacagctggaaaaggaaaagtttcaagaataactccacttctagatactggtgctgcagttacaacttctagaaatgaagtagattatgtagttactgaatatggtgttgctcatcttaagggcaaaactttaagaaatagggcaagagctctaataaatatcgctcatccaaaattcagagaatcattaatgaatgaatttaaaaagagattttag

SEQ ID NO: 14 - DNA, 1071 bp, Clostridium carboxidivorans; identical to SEQ ID NO: 9

SEQ ID NO: 15 - DNA, 1139 bp, Clostridium carboxidivorans
aaaaggttgtgccgcaatatttttaaattgcgaaagttgagtaattaacttaaaaacaggaggaatagtaaatgagttataagatattagcaattaacccaggatctacttctacaaaaatagctttatacgaagatgaaaaagaaatattttgcaaaacgttagagcatccagttgaacaaattgaaaaatatgagaatgtggcagatcaatttgatatgagaaaagaagttgttctttcatttttaaagcaaaatggatatgaagttaaagaattagctgcagttgttggaagaggtggaatggttccaaaagtaaaatctggagcttataaagttaatgaaacaatggtagatagattaaaaaataatccagtagtagaacatgcttcaaatttaggagctttaattgcttatgaaatagcaaattctattggagtatcagcctatatatatgactctgttagagtagatgaattagaggatatagctcgtatatcaggtatgccggatataccaagaacaagtactagtcatgcattaaatacaagggcaatggcaatgaaggttgcaaaaaattatggtaaaaagtattcagatatgaactttattgtagctcatctaggtggaggaatatcagtaaatgttcatagaaaaggacaaatggtagatataatggcagatgacgaaggaccattttcacctgaaagagctggaaaagttccttgcaatgcacttatagatctttgctattcaggaaaatttgataaaaaaactacgaagaaaaaattaaggggaaatggtggattaaaagcttatcttaacactgttgatgctagagaagttgaaagaatgattgaaagtggagatgaaaaagcaaagcttgtttatgaagctatggcttatcaggttgctaagggaataggagaacttgcaacagtagtagaaggtaaggttgatgctatcgttattacaggaggtatagcatattctgatatgataactaactggattaaaaagcgtgtagagtttattgcgcctgttgagattatgcctggtgaaaatgaaatggaatctttggctttgggaactcttagagtgttaaagggtgaagaagaagcaagagaatatgttgaataa

SEQ ID NO: 16 - DNA, 1133 bp, Clostridium carboxidivorans
tttaatatatggcatgatacttgctaattaaaatatttattaagtattgctataatcaggagggtaaaataatgtacaaaatactagcaataaatccaggttcaacttcaactaaaatagctatttatgatgacacagaggaattatttaaaaccactatagaacattctagtgaagaagtgaaaaaatatgaaaacatagctgatcaatatagtatgagatatgaagctataatgaaatttttaaaagaagtagattttgatgtcaaagctttatctgcagtagttggaagaggaggaattctgcctccagttaaatcaggagcttacagagtaaatgattctatggtagaaagactggctaaaagacctgtagtagagcatgcttcaaatttaggagctataatttcatatgcaatagcaaaacctttaaatataccagctttcatatatgattctgtagctgtagatgaatttgaggatattgcaagaatatcaggacttgcagatataaaaagagagagttttattcatgctttaaatatgagagctgcagcaataaaaacagcaaaaaaactaggtaaaccttatgaacaatgtaatttagttgttgctcatttaggaggcggaatatctcttactgtacataaaggtggaaaaatgatagacgctgttactgatgaagaaggaccgttttcaccagaaaggtcaggtagagtaccttgtaagcgcttaatagaaatgtgttataaaaatgatgaacgcacaatgaaaaagaaaataagaggagatggtggattaatctcttatttaggaactaatagtgcattagatgtagaaaaaagaattgaaaatggagatgctgaagccaaattagtttatgaagctatggcatatcaaattgcaaaagcaataggagaacttgcaactgtagtaaagggaaaggttgatgcagtagtaattacagggggaattgcctattcaaaaatgatgacaggatggataaaagaaagagtagaatttatagcacctgtagagatattgccaggagaaaatgaattagaatctcttgctttaggtacgcttagagttataaagggagaagaaaaagcacacgaatatgatttagattag

SEQ ID NO: 17 - DNA, 1056 bp, Clostridium ljungdahlii; identical to SEQ ID NO: 1

SEQ ID NO: 18 - DNA, 1056 bp, Clostridium coskatii
atgaaaggttttgcaatgttaggtattaacaaattaggatggattgaaaagaaaaacccagtgccaggtccttatgatgcgattgtacatcctctagctgtatccccatgtacatcagatatacatacggtttttgaaggagcacttggtaatagggaaaatatgattttaggccatgaagctgtaggtgaaatagccgaagttggcagcgaagttaaagattttaaagttggcgatagagttatcgtaccatgcacaacacctgactggagatctttagaagtccaagctggttttcagcagcattcaaacggtatgcttgcaggatggaagttttccaattttaaagatggtgtatttgcagattactttcatgtaaacgatgcagatatgaatcttgccatactcccagatgaaatacctttagaaagtgcagttatgatgacagacatgatgactactggttttcatggagcagaacttgcagacataaaaatgggctccagcgttgtagtaattggtataggagctgttggattaatgggaatagccggttccaaacttcgaggagcaggcagaattatcggtgttggaagcagacctgtttgtgttgaaacagctaaattttatggagcaactgatattgtaaattataaaaatggtgatatagttgaacaaatcatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttaccaatacctcgtgttcaatggggctgcggcatggctcacaaaactataagaggaggattatgccccggcggacgtcttagaatggaaatgctaagagatcttgttctatataaacgtgttgatttgagtaaacttgttactcatgtatttgatggtgcagaaaatattgaaaaggcccttttgcttatgaaaaataagccaaaagatttaattaaatcagtagttacattctag

SEQ ID NO: 19 - DNA, 1056 bp, Clostridium ragsdalei
atgaaaggttttgcaatgttaggtattaacaagttaggatggattgaaaagaaaaacccagtaccaggtccttatgatgcgattgtacatcctctagctgtatccccatgtacatcagatatacatacggtttttgaaggagcacttggtaatagggaaaatatgattttaggtcacgaagctgtaggtgaaatagctgaagttggcagtgaagttaaagattttaaagttggcgatagagttatcgtaccatgcacaacacctgactggagatccttagaagtccaagctggttttcaacagcattcaaacggtatgcttgcaggatggaagttttccaattttaaagacggtgtatttgcagattactttcatgtaaacgatgcagatatgaatcttgcaatacttccagatgaaatacctttagaaagtgcagttatgatgacagacatgatgactactggttttcatggggcagaacttgctgacataaaaatgggttccagtgttgtcgtaattggtataggagctgttggattaatgggaatagccggttccaaacttcgaggagcaggtagaattatcggtgttggaagcagacccgtttgtgttgaaacagctaaattttatggagcaactgatattgtaaattataaaaatggtgatatagttgaacaaataatggacttaactcatggtaaaggtgtagaccgtgtaatcatggcaggcggtggtgctgaaacactagcacaagcagtaactatggttaaacctggcggcgtaatttctaacatcaactaccatggaagcggtgatactttgccaatacctcgtgttcaatggggctgcggcatggctcacaaaactataagaggagggttatgtcccggcggacgtcttagaatggaaatgctaagagaccttgttctatataaacgtgttgatttgagcaaacttgttactcatgtatttgatggtgcagaaaatattgaaaaggcccttttgcttatgaaaaataagccaaaagatttaattaaatcagtagttacattctaa

SEQ ID NO: 20 - DNA, 146 bp, Clostridium kluyveri; identical to SEQ ID NO: 6

SEQ ID NO: 21 - DNA, 146 bp, Clostridium carboxidivorans
aaaaaggatttaggtatacattcagaaatgatatctgatggtgtagttgacttggtagaatcaggtgtaattaacaataaggaaaaaactttgaatccaggaaaaatagtggtaaccttctttatgggtacaaaaaagctgtatga

SEQ ID NO: 22 - DNA, 101 bp, Clostridium kluyveri; identical to SEQ ID NO: 7

SEQ ID NO: 23 - DNA, 101 bp, Clostridium carboxidivorans
gatggttctacacttcagcttggcataggtgctattccagacgcagtattgttatttttaaaagataaaaaggatttaggtatacattcagaaatgatatc

SEQ ID NO: 24 - DNA, 180 bp, Clostridium carboxidivorans; identical to SEQ ID NO: 8

SEQ ID NO: 25 - DNA, 180 bp, Clostridium carboxidivorans
aaagaacaggaggacttcctgtaggaggtttaataaagctttgctatagtggaaaatatactttagaagaaatgaagaaaaagataagtggaaaaggtggaattgtagcttatctaaatacaaatgattttagggaagtagaacaaaaagcagaaagtggagataaaaaggcaaagttag

SEQ ID NO: 26 - DNA, 20 bp, Artificial Sequence
  Primer
aagcggtgatactttaccaa

SEQ ID NO: 27 - DNA, 20 bp, Artificial Sequence
  Primer
gggccttttcaatattttct

SEQ ID NO: 28 - DNA, 23 bp, Artificial Sequence
  Primer
aaaaaggatytdggkatwcattc

SEQ ID NO: 29 - DNA, 23 bp, Artificial Sequence
  Primer
tcataharyytyttwgtwcccat

SEQ ID NO: 30 - DNA, 20 bp, Artificial Sequence
  Primer
aaagagctggaaaagttcct

SEQ ID NO: 31 - DNA, 20 bp, Artificial Sequence
  Primer
caagctttgctttttcatct